(19) United States
(12) Patent Application Publication
(10) Pub. No.: US 2019/0167527 A1
(43) Pub. Date: Jun. 6, 2019

(54) TOPICAL ANTIMICROBIAL COMPOSITIONS AND METHODS OF FORMULATING THE SAME

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 16/270,335

(22) Filed: Feb. 7, 2019

Related U.S. Application Data
(63) Continuation-in-part of application No. 15/976,579, filed on May 10, 2018.
Application No. 15/976,579 is a continuation-in-part of application No. 14/990,168, filed on Jan. 7, 2016.
Application No. 14/990,168 is a continuation-in-part of application No. 15/597,936, filed on May 17, 2017, now Pat. No. 10,105,342.
Application No. 15/597,936 is a continuation-in-part of application No. 15/668,184, filed on Aug. 3, 2017.
Application No. 15/597,936 is a continuation-in-part of application No. 15/440,800, filed on Feb. 23, 2017.
Application No. 15/440,800 is a continuation-in-part of application No. 14/975,172, filed on Dec. 18, 2015, now Pat. No. 9,707,229.
Application No. 14/975,172 is a continuation-in-part of application No. 14/819,342, filed on Aug. 5, 2015.
(60) Provisional application No. 62/298,994, filed on Feb. 23, 2016.
(60) Provisional application No. 62/298,991, filed on Feb. 23, 2016.
(60) Provisional application No. 62/370,571, filed on Aug. 3, 2016.

(51) Int. Cl. A61J 7/00; A61K 9/00; A61K 9/06; A61K 31/4196; A61K 31/506; A61K 31/5355; A61K 31/496; A61P 31/04; A61P 31/10

(52) U.S. Cl. CPC A61J 7/0007; A61K 9/0014; A61K 9/06; A61K 31/4196; A61P 31/10; A61K 31/5355; A61K 31/496; A61P 31/04; A61K 31/506

(57) ABSTRACT

A method of formulating a topical composition may include combining a powder including an antimicrobial pharmaceutical drug and a topical carrier. The powder may include at least a portion of a crushed oral tablet. The crushed oral tablet may include at least a portion of the antimicrobial pharmaceutical drug. The antimicrobial pharmaceutical drug may be selected from voriconazole, fluconazole, linezolid, levofloxacin, ciprofloxacin, or combination thereof.

### CROSS-REFERENCE TO RELATED APPLICATIONS

The present application is a continuation-in-part of U.S. patent application Ser. No. 15/976,579, filed May 10, 2018, which is hereby incorporated herein by reference. U.S. patent application Ser. No. 15/976,579 is a continuation-in-part of U.S. patent application Ser. No. 14/990,168, filed Jan. 7, 2016, U.S. patent application Ser. No. 15/597,936, filed May 17, 2017, and U.S. patent application Ser. No. 15/668,184, filed Aug. 3, 2017, each of which is hereby incorporated herein by reference, U.S. patent application Ser. No. 15/597,936 is a continuation-in-part application of U.S. patent application Ser. No. 15/440,800, filed Feb. 23, 2017, U.S. patent application Ser. No. 14/975,172, filed Dec. 18, 2015 (now U.S. Pat. No. 9,707,229), and U.S. patent application Ser. No. 14/819,342, filed Aug. 5, 2015, each of which is hereby incorporated herein by reference. U.S. patent application Ser. No. 15/440,800 claims priority under 35 U.S.C. § 119(e) to U.S. Provisional Patent Application No. 62/298,991, filed Feb. 23, 2016, and U.S. Provisional Patent Application No. 62/298,994, filed Feb. 23, 2016, each of which is hereby incorporated herein by reference. U.S. patent application Ser. No. 15/668,184 claims priority under 35 U.S.C. § 119(e) to U.S. Provisional Patent Application No. 62/370,571, filed on Aug. 3, 2016, which is hereby incorporated herein by reference.

### TECHNOLOGY FIELD

The present application relates to antimicrobial compositions, methods of formulating antimicrobial compositions, and methods of using antimicrobial compositions to treat or prevent an infection.

### BACKGROUND

The body normally serves as host for a variety of bacteria and fungi. Most of the time, the balance between the body as host and the microorganisms is maintained. However, there are times when the physiological, biochemical, and/or environmental conditions permit the microorganisms to tip that balance, thereby causing an infection.

Bacterial skin infections include erysipelas, impetigo, MRSA, cellulitis, folliculitis, carbuncles, and hidradenitis suppurativa. These infections can be painful, unsightly, and difficult to treat. Fungal skin infections include thrush, ringworm, yeast infections, nail infections, and diaper rash. Similar to bacterial infections, fungal skin infections can be painful, unsightly, and difficult to treat.

Feet and hands may be infected with bacteria or fungus. These infections present difficult issues for physicians to treat because of the biomechanical complexities of the extremity and the underlying circumstances that cause the infections. Soft tissue infections in the foot consist of any infectious process affecting the skin, subcutaneous tissue, adipose tissue, superficial or deep fascia, ligaments, tendons, tendon sheaths, joints, and/or joint capsules. Considering that there are more than 20 joints, 44 tendons, approximately 100 ligaments, 4 major compartments, and numerous fascial planes in the normal foot, the potential for complex problems is high.

Bacterial infections of the feet can occur as collections of pus, such as an abscess following a puncture wound or an infected hair follicle. These types of infections are usually red and elevated, and sometimes can be mistaken for an insect bite. There are many types of bacteria that cause an abscess, but staph are a leading cause. Bacterial skin infections can also resemble a rash, appearing as a reddened, tender, and warm area of skin. This type of infection is called cellulitis and can spread quickly, leading to red streaks that move from the foot toward the leg. The appearance of streaks is known as lymphangitis, which means the infection is spreading toward the lymph nodes. Cellulitis and lymphangitis can be caused by a variety of types of bacteria, but staph and sometimes streptococcus are the most common causes. Any infection, especially cellulitis and lymphangitis, requires prompt medical attention to avoid further spreading and complications. If left untreated, then some infections can spread to deeper tissues, including bone.

Certain fungal infections of the skin known as tinea infections are caused by dermatophytes, which are members of the Trichophyton, Microsporum, and Epidermophyton species. These mold-like fungi thrive in warm, moist areas, thriving on the dead tissues of hair, nails, and outer skin layers. Tinea infections include tinea pedis, known as athlete's foot; tinea corporis, known as ringworm; tinea capitis, a fungal infection of the scalp that can cause hair loss; tinea cruris, known as jock itch or tinea of the groin; tinea unguum, which is tinea of the nails; and tinea versicolor, a superficial fungal infection that produces brown, tan, or white spots on the trunk of the body. Tinea infections are contagious and can be passed through direct contact or by contact with clothing, from shower and pool surfaces, and even from pets.

Athlete's foot or tinea pedis is by far the most common form, with more than 12 million people in the United States suffering from the disease per year. It presents with redness, itching, burning, cracking, scaling, swelling, and occasionally bleeding. Athlete's foot includes toe web infections, moccasin type infections, and vesicular type infections. The condition generally includes small vesicles, fissures, scaling, maceration, hyperkeratinization, and eroded areas between the toes and on the plantar surface of the foot, as well as on other skin areas. For example, the nails may show thickening, pitting, and subungal debris.

Reoccurrences of the infection are frequent. For some subjects, such as those also diagnosed with diabetes or circulatory problems, or obese subjects, tinea infections and their treatment can be quite serious. The source of the affliction often is a public safety and health concern, as the occurrence of tinea pedis is higher in public areas such as locker rooms, public showers, sports facilities, and the like.

Moreover, there are at least 3 different types of nail infections caused by fungi. The most common infection is frequently caused by Trichophyton rubrum and affects the nail bed and the area beneath the nail. Another type of infection affects only the nail surface and creates white or light colored patches. This second type of fungal infection is unusual and represents only about 10% of the reported cases. A third type of fungal infection affects the nail root and usually afflicts persons with impaired immune defense. A fourth (and unusual) type is caused by an infection of yeast fungi. Infections by yeast most often only affect nails that already are infected or damaged in some way.

The fungi are invasive to the keratin nail tissue. Apart from becoming discolored and brittle, the nail may often separate from the nail bed. In addition, pain and difficulty in wearing foot apparel is often experienced. Initially, the disease affects only one nail, typically one nail of the foot, and is thereafter spread to more nails. The palms of the hands and the soles of the feet may frequently be affected as well. When the skin is affected, red spots frequently occur and the skin may peel off. Nail fungal infections are one of the hardest forms of external infection to treat, of which infections of toe nails are the most difficult to treat.

Despite advances in the understanding of the pathology of bacterial infections and fungal infections, there is still a need for compositions and methods that efficiently treat or prevent the progression and reoccurrence of bacterial infections and fungal infections that affect the skin, nails, and anal and vaginal orifices.

### SUMMARY

In one aspect, a method of formulating a topical composition may include combining a powder including an antimicrobial pharmaceutical drug and a topical carrier. The powder may include at least a portion of a crushed oral tablet. The crushed oral tablet may include the antimicrobial pharmaceutical drug. The antimicrobial pharmaceutical drug may be selected from voriconazole, fluconazole, linezolid, levofloxadin, ciprofloxacin, or combination thereof.

In one embodiment, the method further includes crushing the oral tablet comprising the antimicrobial pharmaceutical drug. In an example, the oral tablet may include a voriconazole 50 mg, 100 mg, or 200 mg oral tablet. In another example, the oral tablet may include a fluconazole 100 mg or 200 mg oral tablet. In still another example, the oral tablet may include a linezolid 600 mg oral tablet. In yet another example, the oral tablet may include a ciprofloxacin 250 mg, 500 mg, or 750 mg oral tablet.

In one example, the oral tablet may include a levofloxacin 250 mg, 500 mg, or 750 mg oral tablet. In another example, the topical carrier comprises a solution, cream, ointment, gel, or foam. In a further example, the topical carrier comprises a commercially available medicated composition comprising at least one additional active pharmaceutical drug.

In one example, the method may include removing a film coating on the oral tablet prior to crushing the oral tablet. Removing the film coating may include agitating the oral tablet for approximately 5 to approximately 20 seconds while the oral tablet is contacted with a solvent, and, thereafter, removing the solvent from contact with the oral tablet.

In another aspect, a method of treating a bacteria or fungal infection includes topically administering a composition that includes at least a portion of an oral tablet combined with a carrier to an affected external skin or nail region or an anal or vaginal orifice. The oral tablet may comprise an antimicrobial pharmaceutical drug selected from voriconazole, fluconazole, linezolid, levofloxacin, ciprofloxacin, or combination thereof.

In various embodiments, the composition comprises a solution. In one example, topically administering the composition comprises administering to skin or a nail of a foot or hand in a footbath. In another example, the composition may comprise an irrigation solution and topically administering the composition includes irrigating the affected external skin or nail region or anal or vaginal orifice. In still another example, the composition comprises a nail lacquer solution and topically administering the composition comprises applying the nail lacquer solution to a nail of a foot or hand.

In one embodiment, the method further includes formulating the composition. Formulating the composition may include combining the carrier and a powder comprising at least a portion of the oral tablet having been crushed to generate the powder. In one example, the oral tablet comprises a voriconazole 50 mg oral tablet, voriconazole 100 mg oral tablet, voriconazole 200 mg oral tablet, fluconazole 100 mg oral tablet, fluconazole 200 mg oral tablet, or combination thereof. In another example, the oral tablet comprises .a linezolid 600 mg oral tablet, ciprofloxacin 250 mg oral tablet, ciprofloxacin 500 mg oral tablet, ciprofloxacin 750 mg oral tablet, levofloxacin 250 mg oral tablet, levofloxacin 500 mg oral tablet, levofloxacin 750 mg oral tablet, or combination thereof.

In various embodiments, the carrier comprises a cream, ointment, gel, foam, or powder. In one example, the carrier comprises a commercially available medicated composition comprising at least one additional active pharmaceutical drug.

In an embodiment, the method further includes crushing the oral tablet and removing a film coating on the oral tablet prior to crushing the oral tablet. In one method, removing the film coating includes agitating the oral tablet for approximately 5 to approximately 20 seconds while the oral tablet is contacted with a solvent, and, thereafter, removing the solvent from contact with the oral tablet.

### BRIEF DESCRIPTION OF THE DRAWINGS

The novel features of the described embodiments are set forth with particularity in the appended claims. The described embodiments, however, both as to organization and manner of operation, may be best understood by reference to the following description, taken in conjunction with the accompanying drawings in which:

FIG. 1 illustrates a pill crushing device according to various embodiments described herein.

### DESCRIPTION

A composition according to the present, disclosure may include a composition formulated to be topically applied to an external surface of a mammal, such as a human. In some embodiments, the composition may be formulated to be administered to skin. Embodiments of the composition may also be formulated to be applied to nails, a vaginal orifice, or anal orifice. Such a composition may be referred to herein as a topical composition.

The topical composition may generally include an antimicrobial agent comprising one or more pharmaceuticals drugs. Some embodiments may include combinations of active agents described herein without the antimicrobial agent. The topical composition may include a carrier comprising one or more carriers, which may be used interchangeably with the term base. The carrier may be liquid, semi-liquid, or solid. For example, the carrier may include an aqueous, organic, or inorganic solution, which may include a dispersion or suspension, cream, gel, ointment, lotion, emulsion, powder, or paste. The topical composition may be formulated to treat microbial infections, such as infections of the skin, nails, mucosal surfaces, and potentially internalized infections, e.g., via transdermal administration of antimicrobial agents.

Embodiments of the topical composition may include an antimicrobial agent selected from an antibacterial agent, antifungal agent, or both. In one embodiment, the antibacterial agent may include an antiviral agent. As introduced above, the topical composition may comprise the antimicrobial agent alone or in combination with one or more additional active agents selected from an antibacterial agent, an antifungal agent, an antiviral agent, an anti-inflammatory agent, a steroid, an anti-allergic agent, an anti-infective agent, an anti-depressant agent, a stimulant agent, a disinfectant agent, an anticonvulsant agent, a local anesthetic agent, or combinations thereof. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antimicrobial agent including an antifungal agent, antibacterial agent, or both alone or in combination with a steroid agent, antiviral agent, NSAID agent, antidepressant agent, anticonvulsant agent, analgesic agent, opiate or opioid agonist agent, keratolytic agent, or combination thereof.

The active agents identified herein may include pharmaceutically acceptable salts and derivatives of the identified active agents.

In various embodiments, the antimicrobial agent comprises an antifungal agent, alone or in combination with an antibacterial agents, wherein the an antifungal agent includes one or more antifungals selected from one or more categories of antifungal agents including azoles (imidazoles), antimetabolites, allylamines, morpholine, glucan synthesis inhibitors (echinocandins), polyenes, benoxaaborale; other antifungal/onychomycosis agents, and new classes of antifungal/onychomycosis agents. For example, the anti-fungal agent may comprise one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In some embodiments, the antibacterial agent is selected from one or more azoles. In one embodiment, the antifungal agent is selected from itraconazole, voriconazole, or combination thereof.

In various embodiments, the topical composition may comprise between approximately 0.01% and approximately 20% by weight antifungal agent, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20%. In some embodiments, the amount of antifungal by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

In various embodiments, the one or more antimicrobial agents comprise an antifungal agent selected from one or more antifungals comprising fluconazole, itraconazole, voriconazole, amphotericin, nystatin, clotrimazole, econazole, or ketoconazole.

In various embodiments, the topical composition comprises the antifungal agent alone or in combination with one or more additional active agents selected from an antibacterial agent, an antiviral agent, an anti-inflammatory agent, an non-steroidal anti-inflammatory (NSAID) agent, an anti-allergic agent, an anti-infective agent, an anti-depressant agent, a stimulant agent, a disinfectant agent, an anticonvulsant agent, a local anesthetic agent, or combinations thereof. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antifungal agent alone or in combination with one or more antibacterial agents, steroid agents, antiviral agents, NSAID agents, antidepressant agents, anticonvulsant agents, analgesic agents, opioid agents, keratolytic agents, or combination thereof. In various embodiments, the topical composition may comprise the antifungal agent alone or in combination with one or more antibacterial agents.

In various embodiments, the antimicrobial agent comprises an antibacterial agent alone or in combination with an antifungal agent. In some embodiments, the antibacterial agent comprises one or more enicillins, cephalosporins, fluoroquinolones, aminoglycosides, monobactams, carbapenems, macrolides, other antibacterial, or combination thereof. For example, the antibacterial agent may include one or more antibacterials selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In some embodiments, the antibacterial agent is selected from mupirocin, gentamycin, tobramycin, or combinations thereof. In one embodiment, the antibacterial agent includes an aminoglycoside.

In various embodiments, the one or more antimicrobial agents comprises an antibacterial agent selected from one or more antibacterials comprising vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole /trimethoprim. In one example, the topical composition comprises linezolid, levofloxacin, ciprofloxacin, or combination thereof

In various embodiments, the topical composition may comprise between approximately 0.01% and approximately 20% by weight, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20%. In some embodiments_{;} the amount of antibacterial by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

In various embodiments, the topical composition comprises the antibacterial agent alone or in combination with one or more additional active agents selected from an antifungal agent, an antiviral agent, an anti-inflammatory agent, an non-steroidal anti-inflammatory (NSAID) agent, an anti-allergic agent, an anti-infective agent, an anti-depressant agent, a stimulant agent, a disinfectant agent, an anticonvulsant agent, a local anesthetic agent, or combinations thereof. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antibacterial agent alone or in combination with one or more antifungal agents, steroid agents, antiviral agents, NSAID agents, antidepressant agents, anticonvulsant agents, analgesic agents, opioid agents, keratolytic agents, or combination thereof In various embodiments, the topical composition may comprise the antibacterial agent alone or in combination with one or more antifungal agents.

As introduced above, the topical composition may comprise one or more additional active agents. It will be appreciated that topical compositions herein may include or specifically exclude additional active agents. It will also be appreciated that topical compositions may exclude an antimicrobial agent and rather include one or more of the additional active agents described herein.

In various embodiments, the topical composition comprises the antimicrobial agent and a nonsteroidal anti-inflammatory drug (NSAID) agent. The NSAID agent may include one or more NSAIDS selected from oxicams, such as meloxicam or piroxicam; salicylic acid derivatives, such as aspirin, diflunisal, salsalate, or trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, or tolmetin; fenamates, such as meclofenamate; and/or COX-2 inhibitors, such as celecoxib, rofecoxib, or valdecoxib. In various embodiments, the topical composition may comprise between approximately 0.01% and approximately 20% by weight NSAID agent.

In various embodiments, the topical composition comprises the antimicrobial agent and a local anesthetic agent. The local anesthetic agent may be selected from lidocaine, prilocaine, benzocaine, or combination thereof. The local anesthetic agent may comprise between approximately 0.01% and approximately 15% by weight of the topical composition.

In various embodiments, the topical composition comprises antimicrobial agent a steroid agent. In one example, the steroid agent comprises a corticosteroid selected from amcinonide, betamethasone dipropionate, betamethasone valerate, clobetasol propionate, desoximetasone, diflorasone diacetate, flurandrenolide, fluticasone propionate, fluocinonide, halcinonide, halobetasol propionate, mornetasone furoate, triamcinolone acetonide, or combination thereof. In various embodiments, the topical composition comprises between approximately 0.001% and approximately 1% by weight steroid agent.

In various embodiments, the topical composition comprises the antimicrobial agent and a muscle relaxant agent comprising one or more muscle relaxants selected from baclofen, carisoprodol, chlorzoxazone, cyclobenzaprine, dantrolene, diazepam, metaxalone, methocarbamol, orphenadrine, quinine sulfate, tizanidine, and/or other muscle relaxants. In various embodiments, the topical composition comprises between approximately 0.001% and approximately 5% by weight muscle relaxant agent.

In various embodiments, the topical composition comprises the antimicrobial agent and an anticonvulsant or nerve depressant agent. The anticonvulsant or nerve depressant agent may comprise one or more nerve depressants and/or anticonvulsants selected from gabapentin, topiramate, lamotrigine, or combinations thereof. In various embodiments, the anticonvulsant or nerve depressant agent may comprise between approximately 0.01% and approximately 20% by weight of the topical composition.

In various embodiments, the topical composition comprises the antimicrobial agent and a NMDA receptor antagonist agent such as ketamine. In some embodiments, the topical composition may comprise an opiate or opioid agonist agent selected from tramadol; one or more C2 opiate agonists selected from oxycodone, morphine, methadone, hydromorphone, and fentanyl; one or more C3 opiate agonists selected from hydrocodone, codeine, propoxyphene, butalbital, and pentazocine; or any combination thereof.

In various embodiments, the topical composition comprises the antimicrobial agents a keratolytic agent selected form urea, salicylic acid, papain, or combinations thereof. For example, the topical composition may comprise the antimicrobial agent and urea. In various embodiments, the topical composition may comprise between approximately 1% and approximately 30% by weight urea.

The topical composition may be provided in a topical format, which may include a carrier for topical administration. In various embodiments, the topical composition may include a colloid or emulsion (o/w, w/o), cream, lotion, ointment, foam, aqueous or non-aqueous gel, aqueous or non-aqueous solution, which may include a dispersion, powder, nail lacquer, bath, or paste.

The topical composition may include a carrier comprising one or more carriers or carrier components thereof The carrier may be liquid, semi-liquid, or solid. For example, the carrier may include an aqueous, organic, or inorganic solution, which may include a dispersion or suspension, cream, gel, ointment, lotion, emulsion, powder, or paste. In some embodiments, the carrier includes a carrier or vehicle composition such as a base cream, ointment, gel, lotion, foam, or solution. The carrier may include carrier components such as lecithin, phospholipids, glycols, paraffin, fatty acids, carbopols/carbomers, alcohols, lanolin, for example.

In some embodiments, the carrier comprises an aqueous solution. In some examples, the carriers comprising aqueous solutions may be combined with the antimicrobial agent to formulate a topical composition comprising an irrigation solution, a footbath, a nail lacquer, a topical spray or soak, for example. In an embodiment, the carrier or component may include an aqueous solution comprising a saline solution. For example, the topical composition may comprise a carrier or component comprising a sodium hydroxide solution, which may be a sterile solution, an alcohol, water, e.g., purified water, water for irrigation, water for injection, or a sterile water. In one embodiment, a carrier or component comprises a sodium chloride 0.09% solution (sterile). The carrier or component may be present in an amount sufficient to obtain the desired amount of active agents per unit weight or volume.

The topical composition may include a carrier comprising a polyethylene glycol (PEG) carrier component. In other embodiments, the composition is PEG-free. In these or other embodiments, the composition may include a silicon or silicon variant carrier component. In some embodiments, the composition is silicon-free. An example topical composition may comprise a solution including carrier components selected from water, alcohol, DMSO, saline or sodium chloride, sodium hypochlorite, or other aqueous or non-aqueous carrier medium into which the one or more active agents are mixed, dispersed, solubilized, or dissolved. The topical composition may be water soluble/miscible or formulated for water absorption. The topical composition may comprise a water-in-oil emulsion or oil-in-water emulsion. In one embodiment, the topical composition comprises a emulsion, e.g., a cream or lotion format, comprising one or more carrier components selected from of acrylate copolymer, alcohol, camphor, carbomer, dimethyl isosorbide, disodium EDTA, dl-alphatocopheryl acetate, edetate disodium, emulsifying wax, eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof. In some embodiments, the topical composition comprises a foam format that includes propellant carrier component such as butane. Topical compositions comprising a foam format may also comprise additional characteristics such as that of an emulsion, such as an oil-in-water emulsion, or gel.

In one example, the topical composition comprises an ointment format and includes active agents in a carrier comprising carrier components selected from hydrophilic petrolatum, white petrolatum, hydrophilic ointment, white ointment, anhydrous lanolin, hydrous lanolin, PEG ointment, or combinations thereof. In an embodiment, the topical composition comprises a gel format. The gel may be an aqueous or non-aqueous gel. The gel may include carrier components thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof. The topical composition may include a powder format and include carrier components such as lactose or talc, for example.

The topical composition or carrier thereof may include carrier components such as one or more solubilizers, stabilizers, buffers, tonicity modifiers, bulking agents, viscosity enhancers/reducers, surfactants, chelating agents, adjuvants, or combinations thereof.

In various embodiments, the topical composition or carrier thereof comprises one or more glucose polymers such as a starch, cellulose, polydextrose, or combination thereof. Example starches may include sodium starch glycolate, corn starch, pregelatinized starch, or combination thereof. Example celluloses may include hydroxypropyl cellulose, hypermellose, croscarmellose sodium, ethyl cellulose, microcrystalline cellulose, or combination thereof. Povidone such as povidone K30, copovidone, crospovidone, or combination thereof, may also be present. In some embodiments, glycol and/or a sugar alcohol may be present. Example glycols may include polyethylene glycol, propylene glycol, or combination thereof. Example sugar alcohols may include mannitol. Some embodiments may include oxides such as silicon dioxide, titanium dioxide, ferric oxide, or combination thereof. One embodiment may include any of the above and magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof. In one embodiment, the topical composition does not include one or more of starch, cellulose, polydextrose, sodium starch glycolate, corn starch, pregelatinized starch, hydroxypropyl cellulose, hypermellose, croscarmellose sodium, ethyl cellulose, microcrystalline cellulose, povidone, povidone K30, copovidone, crospovidone, polyethylene glycol, propylene glycol, mannitol, silicon dioxide, titanium dioxide, ferric oxide, magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof.

The topical composition may be administered topically by contacting an external surface of the body, which may include a vaginal or anal orifice. The topical composition may be administered in a spray, coat, soak, powder, spread, or the like, for example, suitable to the topical format.

In some embodiments, the topical composition comprises a nail lacquer for direct application to nail tissue. A nail lacquer format may include one or more antimicrobial actives formulated for topical application to nail tissue. In some embodiments, a nail lacquer format may include additives such as thickening agents, plasticizers, polymers, volatile organic compounds, or other additives to promote effective localization of the medication following application. In some embodiments, a nail lacquer format may comprise a solution, which may be a suspension or mixture. In some embodiments, a nail lacquer format may lack traditional lacquer additives. In various embodiments, a nail lacquer format may comprise an aqueous solution formulated to be applied to a nail surface whereon the carrier or component thereof evaporates or is absorbed. In some embodiments, a nail lacquer solution may have a fluid or semi-fluid consistency. In some embodiments, a carrier for a nail lacquer format may be thickened with a viscosity agent to increase viscosity for administration. In some embodiments, a nail lacquer format may comprise a solution comprising a cream, lotion, gel, or ointment.

### Method of Compounding a Topical Composition

In various embodiments, a method of formulating the topical composition comprises combining the antimicrobial agent and a topical carrier.

In an embodiment, the antimicrobial agent includes an antifungal agent comprising one or more antifungal actives.

In various embodiments, the method of formulating the topical composition comprises combining an antimicrobial agent comprising an antifungal agent, alone or in combination with an antibacterial agent, and a carrier wherein the an antifungal agent includes one or more antifungals selected from one or more categories of antifungal agents including azoles (imidazoles), antimetabolites, allylamines, morpholine, glucan synthesis inhibitors (echinocandins), polyenes, benoxaaborale; other antifungal/onychomycosis agents, new classes of antifungal/onychomycosis agents, or combinations thereof. For example, the anti-fungal agent may comprise one or more antifungals selected from abafungin, albaconazole, amorolfin, amphotericin b, anidulafungin, bifonazole, butenafine, butoconazole candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In various embodiments, the one or more antimicrobial agents comprises an antifungal agent selected from one or more antifungals comprising fluconazole, itraconazole, voriconazole, amphotericin, nystatin, clotrimazole, econazole, ketoconazole, or combination thereof. In some embodiments, the antibacterial agent one or more azoles. In one embodiment, the antifungal agent is selected from fluconazole, voriconazole, or combination thereof.

In various embodiments, the method of formulating the topical composition may comprise combining with a carrier an antifungal agent, such as any of the above antifungal actives alone or in combination, in an amount by weight of the topical composition between approximately 0.01% and approximately 20% by weight, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between. approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, between approximately 3% and approximately 5%. between 5% and 10%. between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20%. In some embodiments, the antifungal agent may be combined in an of approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

In various embodiments, the method of formulating the topical composition further comprises combining the antifungal, agent, carrier, and one or more additional active agents selected from an antibacterial agent, an antiviral agent, an anti-inflammatory agent, an non-steroidal anti-inflammatory (NSAID) agent, an anti-allergic agent, an anti-infective agent, an anti-depressant agent, a stimulant agent, a disinfectant agent, an anticonvulsant agent, a local anesthetic agent, or combinations thereof. In one embodiment, the method of formulating the topical composition further comprises combining the antifungal agent, carrier with one or more additional actives selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antifungal agent alone or in combination with one or more antibacterial agents, steroid agents, antiviral agents, NSAID agents, antidepressant agents, anticonvulsant agents, analgesic agents, opioid agents, keratolytic agents, or combination thereof.

In various embodiments, the method of formulating the topical composition comprises combining with a carrier an antimicrobial agent comprising an antibacterial agent, alone or in combination with an antifungal agent, wherein the an antibacterial agent comprises one or more enicillins, cephalosporins, fluoroquinolones, aminoglycosides, monobactams, carbapenems, macrolides, other antibacterial, or combination thereof. For example, the method may include combining one or more antibacterials selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In one method, the antibacterial agent is selected from mupirocin, gentamycin, tobramycin, or combinations thereof. According to one method, the antibacterial agent is one or more aminoglycosides. In still another example method, the antimicrobial agent comprises an antibacterial active selected from vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole/trimethoprim. In yet another example, the method include combining an antibacterial agent comprising linezolid, levofloxacin, ciprofloxacin, or combination thereof and a carrier.

In various embodiments, the method of formulating the topical composition may comprise combining with a carrier an antibacterial agent, including any of the above antibacterial actives alone or in combination, in an amount by weight of the topical composition between approximately 0.01% and approximately 20% by weight, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20%. In some embodiments, the amount of antibacterial by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%. approximately 8%. approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical composition.

In various embodiments, the method of formulating the topical composition further comprises combining the antibacterial agent, carrier, and one or more additional active agents selected from an antifungal agent, an antiviral agent, an anti-inflammatory agent, an non-steroidal anti-inflammatory (NSAID) agent, an anti-allergic agent, an anti-infective agent, an anti-depressant agent, a stimulant agent, a disinfectant agent, an anticonvulsant agent, a local anesthetic agent, or combinations thereof. In one embodiment, the method of formulating the topical composition further comprises combining the antibacterial agent, carrier with one or more additional actives selected from one or more anticonvulsants, nerve depressants, muscle relaxants, NMDA (N-Methyl-D-aspartate) receptor antagonists, opiate or opioid agonists, antidepressants, and/or other active agents. In some embodiments, the topical composition may comprise the antibacterial agent alone or in combination with one or more antifungal agents, steroid agents, antiviral agents, NSAID agents, antidepressant agents, anticonvulsant agents, analgesic agents, opioid agents, keratolytic agents, or combination thereof.

As introduced above, the method may include combining the carrier and powder containing all or a portion of the antimicrobial agent. For example, one or more antifungal actives and/or antibacterial actives may be obtained from bulk pure powder or powder for injection and combined with the carrier. In one of an above or another example, one or more actives of the antimicrobial agent may be obtained from one or more commercially available oral tablets. The oral tablets may be crushed and the resulting powder may be combined with the carrier.

In addition to antimicrobial active, in various embodiments, the powder of the crushed tablet may include one or more of a glucose polymer, starch, and/or cellulose and one or more additional components such as magnesium stearate, povidone, lactose, glycol, oxide, talc, triacetin, or an alcohol. In some embodiments, the powder includes a cellulose such as microcrystalline cellulose and one or more of magnesium stearate, anhydrous dibasic calcium phosphate, or povidone. In one example, the powder may further include croscarmellose sodium. In a further example, the powder may also include an oxide such as silicon dioxide, ferric oxide, aluminum oxide, or combination thereof; a starch, such as sodium starch glycolate, corn starch, or both; sodium lauryl sulfate, lactose, talc, or combinations thereof. In some embodiments, wherein the tablet includes a film coating, the method may include removal of all or a portion of a film coating.

In one example, the antimicrobial agent comprises an antifungal agent comprising voriconazole and a method of formulating the topical composition comprises addition of a crushed voriconazole tablet to a carrier. Less than all the powder of a crushed tablet may be used when the tablet contains more voriconazole than required. More than one crushed tablet may be used when the method includes formulating a topical composition comprising more voriconazole than is in the tablet. The voriconazole tablets may comprise commercially available voriconazole 50 mg, 100 mg, 200 mg oral tablets. In some embodiments, other strength tablets may be used. In addition to voriconazole the powder may include a glucose polymer and one or more additional components such as magnesium stearate, povidone, lactose, glycol, oxide, talc, triacetin, or an alcohol. In one example, the powder includes croscarmellose sodium, lactose monohydrate, magnesium stearate, povidone and pregelatinized starch. In a further example, the powder may include hypromellose, lactose monohydrate, polyethylene glycol, talc and titanium dioxide. In another example, the powder may include a starch such as pregelatinized starch, a cellulose such as croscarmellose sodium and/or hypromellose. The powder may also include one or more of lactose monohydrate, magnesium stearate, povidone, titanium dioxide, or triacetin. In another example, the powder may include a starch, croscarmellose sodium, lactose monohydrate, magnesium stearate, polyethylene glycol, polyvinyl alcohol, povidone, talc, and titanium dioxide. In another example, the powder may further include talc. In one example, the powder includes lactose monohydrate, pregelatinized starch (corn), croscarmellose sodium, povidone, magnesium stearate and a coating containing polyvinyl alcohol-part hydrolyzed, titanium dioxide, macrogol/PEG and talc. In one embodiment, the powder may include pregelatinized starch, croscarmellose sodium, lactose monohydrate, magnesium stearate, povidone, and a coating containing hypromellose, lactose monohydrate, titanium dioxide and triacetin.

In various embodiments, the method may comprise crushing one or more voriconazole tablets into a powder. Methods may include crushing 50 mg, 100 mg, or 200 mg voriconazole tablets, for instance. In some embodiments, powder obtained from a crushed voriconazole tablet may be mixed with components of a carrier composition and thereafter additional components may be added to formulate the topical composition as described in more detail elsewhere herein.

In an embodiment, powder obtained from the crushed tablet may be added to a carrier for compounding, such as a base for compounding, or a commercially available medicated composition.

In one example, the antimicrobial agent comprises an antifungal agent comprising fluconazole and a method of formulating the topical composition comprises addition of a crushed fluconazole tablet to a carrier. Less than all the powder of a crushed tablet may be used when the tablet contains more fluconazole than required. More than one crushed tablet may be used when the method includes formulating a topical composition comprising more fluconazole than is in the tablet. The fluconazole tablets may comprise commercially available voriconazole 100 mg or 200 mg oral tablets. In some embodiments, other strength tablets may be used. In addition to fluconazole the powder may include a glucose polymer and one or more additional components such as magnesium stearate, povidone, lactose, glycol, oxide, talc, triacetin, or an alcohol. In some embodiments, the powder includes a cellulose such as microcrystalline cellulose and one or more of magnesium stearate, anhydrous dibasic calcium phosphate, or povidone. In one example, the powder may further include croscarmellose sodium. In a further example, the powder may also include an oxide such as silicon dioxide, ferric oxide, aluminum oxide, or combination thereof; a starch, such as sodium starch glycolate, corn starch, or both; sodium lauryl sulfate, lactose, and talc.

In various embodiments, the method may comprise crushing one or more fluconazole tablets into a powder. Methods may include crushing 100 mg or 200 mg fluconazole tablets, for instance. In some embodiments, powder obtained from a crushed voriconazole tablet may be mixed with components of a carrier composition and thereafter additional components may be added to formulate the topical composition as described in more detail elsewhere herein.

In an embodiment, powder obtained from the crushed tablet may be added to a carrier for compounding, such as a base for compounding, or a commercially available medicated composition.

In one example, the antimicrobial agent comprises an antibacterial agent comprising linezolid and a method of formulating the topical composition comprises addition of a crushed linezolid tablet to a carrier. Less than all the powder of a crushed tablet may be used when the tablet contains more linezolid than required. More than one crushed tablet may be used when the method includes formulating a topical composition comprising more linezolid than is in the tablet. The linezolid tablets may comprise commercially available linezolid 600 mg oral tablets, for example. In some embodiments, other strength tablets may be used. In addition to linezolid the powder may include a glucose polymer comprising a starch and/or a cellulose and one or more additional components such as magnesium stearate, povidone, lactose, glycol, oxide, talc, triacetin, an alcohol, or combination thereof. In various examples, the powder includes a starch and a cellulose. In other embodiments, the powder does not include a starch. In one example, the powder includes croscarmellose sodium, diethyl phthalate, ethyl cellulose, pregelatinized starch, sodium starch glycolate, mannitol, colloidal silicon dioxide, povidone, copovidone, cospovidine, sodium stearyl fumarate, hypromellose, polyethylene glycol, titanium dioxide, magnesium stearate, microcrystalline cellulose, talc, hydroxypropyl cellulose, polydextrose, triacetin, carnauba wax, lactose monohydrate, polacrilin potassium, sodium lauryl sulfate, or a combination thereof. In one example, the powder includes a starch comprising pregelatinized starch, a cellulose comprising hypromellose, a sugar alcohol comprising mannitol, a glycol comprising polyethylene glycol, an oxide comprising titanium dioxide and/or colloidal silicon dioxide, a povidone comprising copovidone, and sodium stearyl fumarate. In another example, the powder includes a cellulose comprising croscarmellose sodium, ethyl cellulose, hypromellose, and/or microcrystalline cellulose, magnesium stearate, povidone, an oxide comprising silicon dioxide and/or titanium dioxide, talc, and diethyl phthalate. In yet another example, the powder comprises a cellulose comprising microcrystalline cellulose. hydroxypropyl cellulose, and/or hypromellose, polydextrose, magnesium stearate, crospovidone, polyethylene glycol, titanium dioxide, and triacetin. In one embodiment, the powder comprises a starch comprising cornstarch and/or sodium starch glycolate, a cellulose comprising microcrystalline cellulose, hypromellose, and/or hydroxypropylcellulose, magnesium stearate, polyethylene glycol, titanium dioxide, and carnauba wax. In another example, the powder comprises hypromellose, lactose monohydrate, magnesium stearate, polyethylene glycol, colloidal silicon dioxide, titanium dioxide, polacrilin potassium, and carnauba wax. In one embodiment, the powder comprises a cellulose comprising croscarmellose sodium and/or hypromellose, lactose monohydrate, magnesium stearate, polyethylene glycol 400, povidone and titanium dioxide. In another embodiment, the powder comprises a cellulose comprising croscarmellose sodium and/or microcrystalline cellulose, polydextrose, magnesium stearate, polyethylene glycol, sodium lauryl sulfate, colloidal silicon dioxide, titanium dioxide and triacetin.

In some embodiments, the powder obtained from a crushed linezolid tablet may be mixed with components of a base or carrier composition and thereafter additional components may be added to formulate the topical composition. In an embodiment, powder obtained from the crushed tablet may be added to a base or carrier for compounding or a commercially available medicated composition as described in more detail elsewhere herein.

In various embodiments, the method may comprise crushing one or more linezolid tablets into a powder. Methods may include crushing 600 mg linezolid tablets, for instance. In some examples, other strength tablets may be used.

In one example, the antimicrobial agent comprises an antibacterial agent comprising levofloxacin and a method of formulating the topical composition comprises addition of a crushed levofloxacin tablet to a carrier. Less than all the powder of a crushed tablet may be used when the tablet contains more levofloxacin than required. More than one crushed tablet may be used when the method includes formulating a topical composition comprising more levofloxacin than is in the tablet. The levofloxacin tablets may comprise commercially available levofloxacin 250 mg, 500 mg, or 750 mg oral tablets, for example. In some embodiments, other strength tablets may be used. In addition to levofloxacin the powder may include a glucose polymer comprising a starch and/or a cellulose and one or more additional components such as magnesium stearate, povidone, lactose, glycol, oxide, talc, triacetin, an alcohol, or combination thereof. In one example, the powder includes cornstarch, croscarmellose sodium, hypromellose, microcrystalline cellulose, magnesium stearate, polyethylene glycol, povidone and titanium dioxide. In another example, the powder includes sodium starch glycolate, hypromellose, microcrystalline cellulose, magnesium stearate, polyethylene glycol, propylene glycol, povidone, polysorbate, colloidal silicon dioxide, and titanium, dioxide. In still another example, the powder includes hypromellose, microcrystalline cellulose, Magnesium stearate, polyethylene glycol 6000, crospovidone, talc and titanium dioxide. In yet another example, the powder includes sodium starch glycolate, croscarmellose sodium, hydroxypropyl cellulose, hypromellose, polyethylene glycol 400, povidone K 30, glycerol behenate, lactose monohydrate, colloidal silicon dioxide, titanium dioxide, ferric oxide, and talc.

In some embodiments, the powder obtained from a crushed levofloxacin tablet may be mixed with components of a base or carrier composition and thereafter additional components may be added to formulate the topical composition. In an embodiment, powder obtained from the crushed tablet may be added to a base or carrier for compounding or a commercially available medicated composition as described in more detail elsewhere herein.

In various embodiments, the method may comprise crushing one or more levofloxacin tablets into a powder. Methods may include crushing 250 mg, 500 mg, 750 mg levofloxacin tablets, for instance. In some examples, other strength tablets may be used.

In one example, the antimicrobial agent comprises an antibacterial agent comprising ciprofloxacin and a method of formulating the topical composition comprises addition of a crushed ciprofloxacin tablet to a carrier. Less than all the powder of a crushed tablet may be used when the tablet contains more ciprofloxacin than required. More than one crushed tablet may be used when the method includes formulating a topical composition comprising more ciprofloxacin than is in the tablet. The levofloxacin tablets may comprise commercially available ciprofloxacin hydrochloride 250 mg, 500 mg, or 750 mg oral tablets for example. In some embodiments, other strength tablets may be used. In addition to ciprofloxacin the powder may include the powder may include a glucose polymer and one or more additional components such as magnesium stearate, povidone, lactose, glycol, oxide, talc, triacetin, or an alcohol. In one example, the powder includes a starch such as cornstarch, sodium starch glycolate. The powder may also include magnesium stearate and/or lactose. In one embodiment, the powder includes a cellulose such as croscarmellose sodium and/or microcrystalline cellulose. The powder may also include magnesium stearate, povidone, and/or and oxide such as silicone dioxide. In an embodiment, the powder includes a cellulose such as hypromellose and/or microcrystalline cellulose. The powder may also include a starch such as cornstarch and/or sodium starch glycolate. In a further example, the powder also includes magnesium stearate. In still a further example, the powder includes polydextrose, silicon dioxide, titanium dioxide, talc, and/or triacetin. The powder may also include polyethylene glycol. In one embodiment, the powder includes a cellulose such as microcrystalline cellulose. The powder may also include a starch such as sodium starch glycolate. In a further example, the powder also includes magnesium stearate. In still a further example, the powder includes povidone, silicon dioxide, titanium dioxide, and/or polyethylene glycol. In an embodiment, the powder includes a cellulose such as croscarmellose sodium, hypromellose, and/or microcrystalline cellulose. The powder may also include a starch such as cornstarch. In a further example, the powder also includes magnesium stearate. In still a further example, the powder includes povidone, silicon dioxide, titanium dioxide, talc, and/or carnauba wax. In yet a further example, the powder includes stearic acid, succinic acid, sodium lauryl sulfate, and/or polyvinyl alcohol. The powder may also include polyethylene glycol. In one embodiment, the powder includes a cellulose such as ethylcellulose and/or hypromellose. In a further example, the powder also includes magnesium stearate. In still a further example, the powder includes povidone such as crospovidone, titanium dioxide. In yet a further example, the powder includes succinic acid. The powder may also include polyethylene glycol.

In some embodiments, the powder obtained from a crushed levofloxacin tablet may be mixed with components of a base or carrier composition and thereafter additional components may be added to formulate the topical composition. In an embodiment, powder obtained from the crushed tablet may be added to a base or carrier for compounding or a commercially available medicated composition as described in more detail elsewhere herein.

In various embodiments, the method may comprise crushing one or more ciprofloxacin tablets into a powder. Methods may include crushing 250 mg, 500 mg, 750 mg ciprofloxacin tablets, for instance. In some examples, other strength tablets may be used.

As introduced above, tablets may include film coatings. For example, film coatings may comprise polymer coatings such as a shellac. In some embodiments, the method of formulating the topical composition may include removing all or a portion of the film coating. In one example, the method includes removal of all or a portion of the film coating prior to crushing the tablet. For example, the film coating may be removed by contacting the skin/coating with a solvent. The solvent may include an alcohol, sterile sodium chloride, or aqueous solvent such as water. Contacting may include spraying or pouring the solvent onto the tablet to coat the tablet with the solvent. In one method, contacting includes submerging the tablet in an excess of solvent. The contacted tablet may be shaken with the solvent or may be allowed to rest for a sufficient period of time for solvent to act. According to one method, the contacting may be brief such as less than a minute, less than approximately 30 seconds, less than approximately 20 seconds, or between approximately 5 seconds and approximately 20 seconds. In one example, tablets coated in solvent may be quickly washed to remove the film coating after the solvent has contacted the film coating for a suitable period of time. In another method, a tablet including a film coating may be crushed and thereafter mixed with water or solvent, the powder including the film coating may then go into solution. According to a version of the method, the powder and water or solvent may be shaken to accelerate the powder going into solution.

In various embodiments, the method may include crushing one or more tablets with a pill crushing device. It will be understood that tablets may be crushed without utilizing a pill crushing device by compressing and/or grinding the tablet.

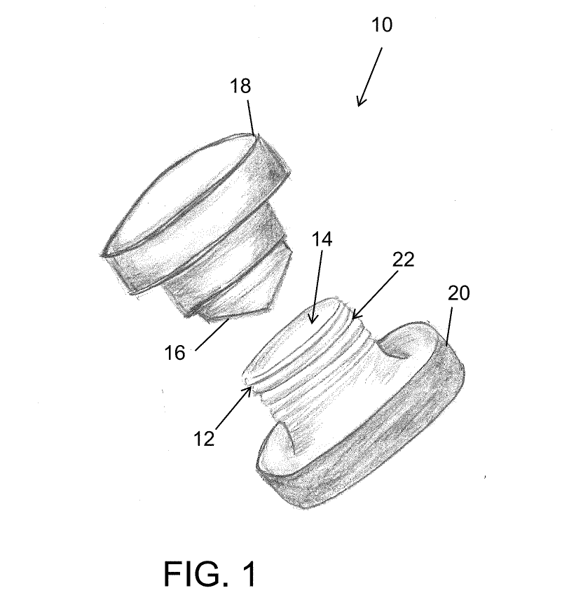

FIG. 1 illustrates a pill crushing device 10 for crushing tablets according, to various embodiments. The pill crushing device 10 includes a container portion 12 defining an interior volume 14 into which one or more tablets may be inserted. A crushing member 16 comprising a projection is dimensioned to be inserted into the interior volume 14 of the container portion 12 and worked against the tablet to crush the tablet between the crushing member 16 and an interior wall of the container portion 12 defining the interior volume 14, which may be a bottom or sidewall, for example. The crushing member 16 is disposed on a first component 18 that may be separated from a second component 20 including the container portion 12. The first component 18, second component 20, or both may be configured to be manipulated by a user to crush the tablet when the crushing member 16 is extended into the interior volume 14. For example, a user may rotate the crushing member 16 and compress the tablet therewith by compressing the first component 18 and second component 20 toward each other and rotating the two relative to the other. As shown, the first component 18 and second component 20 of the pill crushing device 10 are configured to threadably engage along threads 22. Similar threads, which are not visible in FIG. 1, may be provided on the first component 18 to mate, with the threads 22 on the second component 20. According to one crushing method, the first component 18 or crushing member 16 thereof may be threaded to second component 20 or container portion 12 thereof along threads 22 such that the threading progressively extends the crushing member 16 into the interior volume 14 of the container portion 12 to crush the tablet therein. For example, the first portion 18 may be threaded such that the crushing member 16 compresses tightly against the tablet(s). The first portion 18 may then be rotated relative to the second portion 20 to crush the tablet and back and forth to generate a uniform powder. In another configuration (not shown), the crushing member 16 may be coupled to the container portion 12 by a hinge and be pivotable thereon into and out from the interior volume 14 of the container portion 12.

In one embodiment, solvent may be added into the interior volume 14 of the container portion 12 (FIG. 1) including the crushed tablet. The pill crushing device 10 may then be shaken to dissolve the film coating prior to crushing the tablet. The pill crushing device 10 may be shaken until the film coating is sufficiently removed, e.g., for less than a minute, less than approximately 30 seconds, less than approximately 20 seconds, or between approximately 5 seconds and approximately 20 seconds. The solvent may be drained to remove the film coating. Other embodiments wherein the film coating is removed prior to crushing the tablet may include shaking the solvent and tablet in a container that is different than the container portion 12 or the pill crushing device 10. In one example, such a container includes a drain for removal of solvent. In one embodiment, such a container includes a sieve or strainer and the solvent may be contacted to the film or coating and allowed to drain from therefrom to remove the film coating. In some embodiments, solvent may be added into the interior volume 14 of the container portion 12 including the crushed tablet powder that does not include a film coating. According to one example, the pill crushing device 10 may be shaken until the film coating is sufficiently removed or until the powder including the film coating is into solution or, in one permutation, a paste is formed. According to one embodiment, the pill crushing device 10 containing the solvent may be shaken for less than a minute, less than approximately 30 seconds, less than approximately 20 seconds, or between approximately 5 seconds and approximately 20 seconds. According one method, the powder from the crushed tablet may be poured into a separate container for mixing with a carrier. In one example, a solvent is not added to the powder prior to addition to the carrier,

The number of tablets to be crushed to formulate a topical composition comprising a desired weight of an active agent may be determined by dividing the weight of the active agent needed by the weight of the active in a tablet. The weight of crushed tablet powder needed to formulate a topical composition comprising a desired weight of an active agent may be determined by dividing the weight of a tablet by the weight of the active agent present in the tablet and multiplying the result by the desired weight of the active agent in the topical composition.

A method of formulating a topical composition may include combining one or more active agents, such as an antimicrobial agent as described herein, and a carrier. In some embodiments, the antimicrobial agent may include pure powder formats of one or more antibacterial and/or antifungal agents combined with the carrier in addition to powder from crushed tablets. Combining may include adding all or a portion of the powder to be combined with all or a portion of the carrier and mixing. In some embodiments, all or a portion of the powder may be dispersed, suspended, or dissolved in a liquid to form a paste, solution, dispersion, or suspension prior to addition to the carrier. In one of an above or another embodiment, all or a portion of the powder may be directly added to all or a portion of the carrier. According to various embodiments, the carrier may comprise a suitable carrier selected to formulate a topical composition comprising a format selected from a cream, gel, lotion, ointment, emulsion (oil-in-water or water-in-oil), foam, solution, dispersion, or powder, for example, suitable for topical application. The carrier may be present in an amount sufficient to obtain the desired amount of active agents per unit weight or volume. The one or more active agents may be mixed dispersed, suspended, solubilized, or dissolved with the carrier.

In some embodiments, the method includes formulating a carrier and/or combining the active agent with a carrier comprising a base cream, ointment, gel, lotion, foam solution, or powder. The carrier may include lecithin, phospholipids, glycols, paraffin, fatty acids, carbopols/carbomers, alcohols, lanolin, or combination thereof, for example. In one embodiment, the carrier comprises a sodium chloride 0.09% solution (sterile). Some embodiments may include polyethylene glycol (PEG), while other embodiments may be PEG-free. In an above embodiment or another embodiment, the carrier may include a silicon or silicon variant or may be silicon-free. A carrier solution may comprise an aqueous or non-aqueous solution. Example solutions may include water, alcohol, DMSO, saline or sodium chloride, and/or sodium hypochlorite. In some embodiments, the carrier comprises an aqueous solution such as a saline solution. For example, the topical composition may comprise a carrier comprising sodium hydroxide solution, which may be a sterile solution, an alcohol, water, e.g., purified water, water for irrigation, water for injection, or a sterile water. The carrier may be water soluble/miscible or formulated for water absorption, such as a gel.

In some embodiments, the method includes combining the active agent with a carrier to formulate a topical composition comprising a water-in-oil emulsion or oil-in-water emulsion. For example, the carrier may comprise an emulsion having a cream or lotion format including one or more of acrylate copolymers, alcohol, camphor, carbomer, dimethyl isosorbide, disodium EDTA, dl-alphatocopheryl acetate, edetate disodium, emulsifying wax, eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof.

In some embodiments, the method includes combining the active agent with a carrier to formulate a topical composition comprising an ointment format. For example, the carrier may comprise hydrophilic petrolatum, white petrolatum, hydrophilic ointment, white ointment, anhydrous lanolin, hydrous lanolin, PEG ointment, or combinations thereof. In some embodiments, the method includes combining the active agent with a carrier to formulate a topical composition comprising a gel. The gel may be an aqueous or non-aqueous gel. The gel may include thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof In some embodiments, the method includes combining the active agent with a carrier to formulate a topical composition comprising a powder. A powder carrier may include lactose or talc, for example. In some embodiments, the method may include imparting the carrier or topical composition formulated therewith with a gas or pressurized propellant to generate a foam format. For example, a propellant such as butane may be used to generate a foam from the carrier or combined carrier and active agent. In various embodiments, the method may include utilizing a carrier or further combining of one or more carrier component additives such as solubilizers, stabilizers (which may include antioxidants), buffers, tonicity modifiers, bulking agents, viscosity enhancers/reducers, surfactants, chelating agents, adjuvants, humectants, preservatives, flavorings, binders, colorants, or combinations thereof.

Further to the above, in some embodiments, the method includes combining the active agent with a commercially available carrier or base vehicle composition for compounding. The carrier may be liquid, semi-liquid, or solid. For example, the carrier may include an aqueous, organic, or inorganic solution, which may include a dispersion or suspension, cream, gel, ointment, lotion, emulsion, powder, or paste. Thus, the method of formulating the topical composition may include addition of crushed antimicrobial tablets or powder thereof to a topical base for compounding to formulate creams, ointments, solutions/irrigations/baths, powders, gels, lotions, or pastes, for example. Non-limiting examples may include Spira-Wash® Gel, Lipoderm®, Loxasperse®, Mucolox™, or Versabase® Cream, Goam, Gel, Lotion or Shampoo, manufactured and distributed by PCCA, 9901 South Wilcrest Drive, Houston, Tex. 77099.

In one embodiment, the method of formulating the topical composition comprises combining all or a portion of the active agent and a carrier comprising a commercially available medicated composition. In one example, the methods includes combining all or a portion of an antimicrobial agent comprising one or more crushed antimicrobial tablets and commercially manufactured medicated composition. The commercially manufactured medicated composition may comprise a cream, ointment, lotion, suspension, dispersion, solution, irrigation, bath, powder, gel, foam, paste, for example. Thus, a method of formulating the topical composition may comprise adding a first portion of the active agent comprising pure powder or crushed tablets to a medicated composition comprising a second portion of the active agent and at least a portion of the carrier. The commercially available composition may comprise a medicated composition for oral administration, topical administration, ophthalmic administration, otic administration, nasal administration, transdermal administration, sublingual administration, or pulmonary administration.

In an embodiment, the method include combining crushed tablets of a portion of the antimicrobial agent to another portion of the antimicrobial agent comprising another format, such as a commercially available medicated suspension, solution, ointment, cream, gel, lotion, or powder. In some such embodiments, the carrier may be provided by the commercially available medicated composition. In various embodiments, all or a portion of the powder may be added to one or more components of the carrier. Thereafter, additional carrier components and the remainder of the powder may be added to formulate the topical composition. Other active agents may also be added before, after, or with the powder antimicrobial. It will be appreciated that unless indicated otherwise, combining or adding the powder to the carrier or component thereof may include combining or adding the powder after the powder has been combined or mixed with a liquid or other composition. In one example, combining the powder and carrier results in a formulation of a topical composition having a different format than that of the carrier, such a commercially manufactured base for compounding or commercially manufactured medicated composition. Examples may include addition of additional carrier components such described above such as thickening agents, thinners, surfactants, carbomers, PEG, hydrocarbons, and/or diluents.

In some embodiments, the method includes combining a first portion of the antimicrobial agent comprising crushed tablets with a second portion of the antimicrobial agent. The method may include combining the first and second portions and the carrier together or separately. The antimicrobial agent may be one or more antifungal agents, one or more antibacterial agents, or a combination thereof In one embodiment, the second portion of the antimicrobial agent comprises a commercially available medicated composition.

In one example, a second portion of the antimicrobial agent may comprise all or a portion of an antifungal agent selected from any of the antifungal agents described herein. For instance, a second portion of the antimicrobial agent may comprise all or a portion of an antifungal agent comprising an azole. In one example, the second portion of the antifungal agent may comprising voriconazole bulk powder. In another or further example, a second portion of the antifungal agent may comprise fluconazole bulk powder or powder for suspension. In an above or another example, a second portion of the antimicrobial agent may comprise itraconazole, ketoconazole, econazole, or combination thereof.

In one example, a second portion of the antimicrobial agent may comprise all or a portion of an antibacterial agent selected from any of the antibacterial agents described herein. For instance, a second portion of the antimicrobial agent may comprise all or a portion of an antibacterial agent comprising Linezolid Powder for Injection, Linezolid Powder, or bulk powder. As another or further example, a second portion of the antimicrobial agent may comprise all or a portion of an antibacterial agent comprising levofloxacin bulk powder. In an above or another example, a second portion of the antimicrobial agent may comprise all or a portion of an antibacterial agent comprising ciprofloxacin bulk powder.

As introduced above, the method may include combining a first portion of an antimicrobial agent with a carrier wherein the carrier comprises a commercially manufactured medicated composition comprising a second portion of the antimicrobial agent.

In various embodiments, the method comprises combining an antimicrobial agent and a carrier wherein the antimicrobial agent comprises an antifungal agent.

In an example, the antifungal agent or a carrier comprising at least a portion of the antifungal agent may comprise a commercially available fluconazole, such as Fluconazole in Dextrose Inject, Solution; Fluconazole in Sodium Chloride Injection, Solution; Fluconazole Injection; Fluconazole Powder, for Suspension; Fluconazole Tablets; or bulk powder.

In an example, the antifungal agent or a carrier comprising at least a portion of the antifungal agent may comprise a commercially available itraconazole, such as Itraconazole Capsule; Itraconazole Injection Solution; or bulk powder.

In an example, the antifungal agent or a carrier comprising at least a portion of the antifungal agent may comprise a commercially available amphotericin, such as Amphotericin B injection, Lipid Complex; Amphotericin B Injection, Powder, Lyophilized, for Solution; or bulk powder.

In an example, the antifungal agent or a carrier comprising at least a portion of the antifungal agent may comprise a commercially available econazole such as an econazole nitrate 1.0% cream.

In an example, the antifungal agent or a carrier comprising at least a portion of the antifungal agent may comprise a commercially available fluconazole, for example, as 100 mg or 200 mg tablets.

In an example, the antifungal agent or a carrier comprising at least a portion of the antifungal agent may comprise a commercially available ketoconazole as 50 mg, 100 mg, or 200 mg tablets.

In an example, the antifungal agent or a carrier comprising at least a portion of the antifungal agent may comprise a commercially available nystatin, such as Nystatin Powder (Topical), or bulk powder.

In one embodiment, the carrier may comprise a commercially manufactured medicated composition comprising a second portion of the antimicrobial agent comprising all or a portion of an antifungal agent selected from any of the antifungal agents described herein, such as an azole. The composition may comprise, for example, a commercially available medicated composition comprising an antifungal suspension, solution, dispersion, ointment, cream, gel, lotion, or powder.

In one example, the carrier comprises a commercially available clotrimazole composition such as a Clotrimazole Cream, Clotrimazole Lotion, Clotrimazole Liquid, or Clotrimazole Solution.

In an above or another example, the carrier comprises a commercially available econazole composition, such as Econazole Nitrate Cream or Econazole Nitrate Foam.

In an above or another example, the carrier comprises a commercially available ketoconazole composition such as Ketoconazole Foam, Ketoconazole Cream, Ketoconazole Suspension, or Ketoconazole Suspension Shampoo.

In an above or another example, the carrier comprises a commercially available fluconazole composition such as Fluconazole Cream, Fluconazole in Dextrose Injection Solution; Fluconazole in Sodium Chloride Injection Solution, Fluconazole Injection. Solution, or Fluconazole Suspension.

In an above or another example, the carrier Comprises a commercially available fluconazole composition such as Voriconazole Ophthalmic Ointment or Voriconazole Oral Suspension. The Voriconazole Oral Suspension may include 45 g powder for oral suspension for reconstitution with water to produce a suspension containing 40 mg/mL voriconazole and including colloidal silicon dioxide, titanium dioxide, xanthan gum, sodium citrate dihydrate, sodium benzoate, anhydrous citric acid, natural orange flavor, and sucrose.

In an above or another example, the carrier comprises a commercially available nystatin composition such as Nystatin Cream or Nystatin Ointment.

In various embodiments, the method comprises combining an antimicrobial agent and a carrier wherein the antimicrobial agent comprises an antibacterial agent.

In an example, the antibacterial agent or a carrier comprising at least a portion of the antibacterial agent may comprise vancomycin powder, such as Vancomycin Hydrochloride for Injection, USP, which is a lyophilized powder for preparing intravenous (IV) infusions. The powder may be provided in vials (e.g., bottles) containing the equivalent of 500 mg, 1 g, 5 grams, 10 grams vancomycin base. Other formats may be used, for example Vancomycin Hydrochloride USP powder for oral solution, equivalent to 3.75 g, 7.5 g or 15 g vancomycin, and diluent, which may be a flavored, e.g., grape-flavored, diluent for reconstitution; Vancomycin Intravenous Solution, e.g., vancomycin hydrochloride 5 mg/mL, sodium chloride 9 mg/mL.

In an example, the antibacterial agent or a carrier comprising at least a portion of the antibacterial agent may comprise a commercially available ciprofloxacin, such Ciprofloxacin Hydrochloride Solution/Drops; Ciprofloxacin Hydrochloride Tablets; Ciprofloxacin Tablets, e.g., 500 mg or 100 mg; Ciprolloxacin Hydrochloride Suspension; Ciprofloxacin Injection, USP, e.g., Ciprofloxacin Injection, USP, 20 mL, 200 mg, 1% and 40 ml or 400 mg, 1%, for intravenous injection and infusion, Premix 100 mL in 5% Dextrose, 200 mg, 0.2% and 200 mL in 5% Dextrose or 400 mg, 0.2%, for intravenous infusion; or bulk powder.

In an example, the antibacterial agent or a carrier comprising at least a portion of the antibacterial agent may comprise a commercially available levofloxacin, such as Levofloxacin Injection, which may be supplied in single-use vials containing a concentrated solution with the equivalent of 500 mg of levofloxacin USP in 20 mL vials and 750 mg of levofloxacin USP in 30 mL vials; Levofloxacin Solution/Drops; Levofloxacin Tablet 250 mg, 500 mg, 750 mg; or bulk powder.

In an example, the antibacterial agent or a carrier comprising at least a portion of the antibacterial agent may comprise a commercially available azithromycin, such as Azithromycin for Injection USP, which may be supplied in lyophilized form under a vacuum in a 10 mL vial equivalent to 500 mg of azithromycin for intravenous administration including sodium hydroxide and 413.6 mg citric acid; Azithromycin for Oral Suspension, USP, which may be supplied for suspension in 100 mg/5 mL or 200 mg/5 mL; Azithromycin Tablets; or bulk powder.

In an example, the antibacterial agent or a carrier comprising at least a portion of the antibacterial agent may comprise a commercially available clindamycin, such as Clindamycin Phosphate Cream; Clindamycin Phosphate Gel; Clindamycin Phosphate Suspension; Clindamycin Phosphate Injection Solution; Clindamycin Phosphate for Injection; or bulk powder.

In an example, the antibacterial agent or a carrier comprising at least a portion of the antibacterial agent may comprise a commercially available doxycycline, such as Doxycycline Hyclate tablets; Doxycycline Hyclate Tablets; Doxycycline Hyclate Pellets; Doxycycline for Suspension; Doxycycline Hyclate Powder for Suspension; or bulk powder.

In an example, the antibacterial agent or a carrier comprising at least a portion of the antibacterial agent may comprise a commercially available mupirocin, such as Mupirocin Ointment; Mupirocin Cream; or bulk powder.

In an example, the antibacterial agent or a carrier comprising at least a portion of the antibacterial agent may comprise a commercially available cefepime, such as Cefepime Hydrochloride Injection, Powder, for Solution, supplied in 500 mg, 1 g, and 2 g vials; Cefepime Hydrochloride Injection Solution; or bulk powder.

In an example, the antibacterial agent or a carrier comprising at least a portion of the antibacterial agent may comprise a commercially available streptomycin, such as Streptomycin for Injection USP, which may be supplied in 1 g vials; Streptomycin Injection, Powder, Lyophilized, for Solution; or bulk powder.

In an example, the antibacterial agent or a carrier comprising at least a portion of the antibacterial agent may comprise a commercially available sulfamethoxazole/trimethoprim, such as Sulfamethoxazole and Trimethoprim Tablets; Sulfamethoxazole and Trimethoprim Injection; Sulfamethoxazole and Trimethoprim Suspension; or bulk powder.

In an embodiment, the method may include combining an antimicrobial agent or a portion thereof with a carrier comprising a commercially available antifungal composition comprising combining an antibacterial agent comprising Azithromycin Oral Suspension, Ciprofloxacin Cream, Ciprofloxacin Ointment, Clindamycin Cream, Clindamycin Ointment, Clindamycin Gel, Gentamycin drops, Gentamycin Spray, Gentamycin Cream, Gentamycin Ointment, Levofloxacin Injection Solution, Levofloxacin Drops, Mupirocin Ointment, Mupirocin Cream, Tobramycin Ophthalmic Ointment, Tobramycin Ophthalmic Drops, and/or Tobramycin Otic Drops.

In an aspect, the carrier comprises a commercially available Mupirocin Ointment wherein each gram of mupirocin 2.0% ointment can contain 20 mg mupirocin in a bland water miscible ointment base (polyethylene glycol ointment, NF) comprising polyethylene glycol 400 and polyethylene glycol 3350. In an aspect, mupirocin can be commercially available, for example, as a mupirocin 2.0% ointment. In an aspect, a mupirocin 2.0% ointment may be provided in a tube, such as, for example, a 22 g tube. In an aspect, mupirocin ointment may include mupirocin cream USP containing 2.15% w/w mupirocin calcium USP (equivalent to 2% mupirocin free acid) in an oil- and water-based emulsion supplied in 15-gram and 30-gram tubes.

In one embodiment, the method includes mixing linezolid oral suspension with one or more active agents, such as an antibacterial agent or an antifungal active. An example, oral suspension may include inactive ingredients such as sucrose, citric acid, sodium citrate, microcrystalline cellulose and carboxymethylcellulose sodium, aspartame, xanthan gum, mannitol, sodium benzoate, colloidal silicon dioxide, sodium chloride, or combination thereof.

As introduced above, the method may include combining active agents in addition to the antimicrobial agent. In some embodiments, the additional active agent comprises one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof.

It will be appreciated that topical compositions herein may include or specifically exclude an additional active agent. It will also be appreciated that topical compositions may exclude an antimicrobial agent and rather include one or more of the additional active agents described herein.

The method of formulating the topical composition comprising additional active agents may include combining all or a portion of an additional active agent from a powder format, e.g., from bulk powder, crushed tablet, injection powder, or other commercially available composition. In various embodiments, such additional active agents may be combined with the carrier together with or separate from all or a portion of the antimicrobial agent powder or other format. According to a method, all or a portion of an additional active agent may be provided in a format selected from a solution, emulsion, gel, cream, lotion, ointment, or other format and may be combined with the carrier together with or separate of all or a portion of the antimicrobial agent. In one example, all or a portion of the additional active agent may be mixed with all or a portion of the antimicrobial agent prior to being added to the carrier. In another example, the antimicrobial agent is added to the additional active agent that is provided in a commercially available medicated composition comprising the carrier.

In one embodiment, formulating the topical composition comprising combining the antimicrobial agent with a commercially available medicated composition comprising all or a portion of the additional active agent.

In one embodiment, the method of formulating the topical composition comprises combining an antimicrobial agent, carrier, and a nonsteroidal anti-inflammatory drug (NSAID) agent. The NSAID agent may include one or more NSAIDS selected from oxicams, such as meloxicam or piroxicam; salicylic acid derivatives, such as aspirin, diflunisal, salsalate, or trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, or tolmetin; fenamates, such as meclofenamate; and/or COX-2 inhibitors, such as celecoxib, rofecoxib, or valdecoxib. In various embodiments, the topical composition may comprise between approximately 0.01% and approximately 20% by weight NSAID agent.

In one embodiment, the method of formulating the topical composition comprises combining the antimicrobial agent, an NSAID agent identified herein, and a carrier in an amount sufficient to formulate the topical composition comprising the antimicrobial agent in an amount between approximately 0.01% and approximately 10% by weight, such as between approximately 0.5% and approximately 5% or any other percent, percent range, or percent therebetween by weight described herein and the NSAID agent in an amount between approximately 0.01% and approximately 20% by weight, such as between approximately 2% and approximately 10% or any other percent, percent range, or percent therebetween by weight described herein. The antimicrobial agent may be or include any antibacterial or antifungal active described herein. For example, in one embodiment, the antimicrobial agent comprises an antifungal agent selected from fluconazole, voriconazole, or combination thereof. In one embodiment, the method includes crushing voriconazole 50 mg, 100 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the antimicrobial agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg, tablets and the carrier. Combining the NSAID may comprise adding a bulk powder, crushed tablet, or injection powder. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one embodiment, the carrier comprises a commercially available medicated composition comprising a portion of the antimicrobial or an additional active agent as described herein. In one example, the carrier includes all or a portion of the NSAID agent and includes a commercially available medicated NSAID composition comprising a cream, ointment, suspension, lotion, gel, or solution. For example, the carrier may comprise a commercially available medicated NSAID composition comprising a Diclofenac Sodium Solution. Diclofenac Sodium Solution may contain, for example, 1.5% (w/w), diclofenac sodium wherein each 1 mL of solution contains approximately 16.05 mg of diclofenac sodium. In one embodiment, the diclofenac solution comprises a diclofenac sodium solution, 1.5% (w/w), such as that which is manufactured under the trade name PENNSAID® by Nuvo Manufacturing, Varennes, Quebec, Canada or Diclofenac Sodium Topical Solution, 1.5% (w/w), manufactured by Apotex Inc. Toronto, Ontario, Canada. M9L 119 for Apotex Corp. Weston, Fla. 33326 for treating the pain of osteoarthritis of the knee. The diclofenac solution may also contain various inactive ingredients such as dimethyl sulfoxide USP (DMSO, 45.5% w/w), ethanol, glycerin, propylene glycol and purified water. In one embodiment, the diclofenac solution comprises a diclofenac sodium solution marketed under the trade name PENNSAID® and manufactured by Nuvo Manufacturing, Varennes, Quebec, Canada, in a 2% (w/w) diclofenac solution for treating the pain of osteoarthritis of the knee. Each gram of solution may contain approximately 20 mg of diclofenac sodium and various inactive ingredients such as dimethyl sulfoxide USP (DMSO, 45.5% w/w), ethanol, purified water, propylene glycol, and hydroxypropyl cellulose. In other embodiments, other concentrations of diclofenac solution, such as diclofenac sodium solutions, may be used.

In one embodiment, the method of formulating the topical composition comprises combining an antimicrobial agent, carrier, and a local anesthetic agent. The local anesthetic agent may be selected from lidocaine, prilocaine, benzocaine, or combination thereof. The local anesthetic agent may comprise between approximately 0.01% and approximately 15% by weight of the topical composition.

In one embodiment, the method of formulating the topical composition comprises combining the antimicrobial agent, a local anesthetic agent identified herein, and a carrier in an amount sufficient to formulate the topical composition comprising the antimicrobial agent in an amount between approximately 0.01% and approximately 10% by weight, such as between approximately 0.5% and approximately 5% or any other percent, percent range, or percent therebetween by weight described herein and the local anesthetic agent in an amount between approximately 0.01% and approximately 12% by weight, such as between approximately 2% and approximately 10% or any other percent, percent range, or percent therebetween by weight described herein. The antimicrobial agent may be or include any antibacterial or antifungal active described herein. For example, in one embodiment, the antimicrobial agent comprises an antifungal agent selected from fluconazole, voritonazole, or combination thereof. In one embodiment, the method includes crushing voriconazole 50 mg, 10.0 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the antimicrobial agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg tablets and the carrier. Combining the local anesthetic may comprise adding a bulk powder, crushed tablet, or injection powder. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one embodiment, the carrier comprises a commercially available medicated composition comprising a portion of the antimicrobial agent or an additional active agent as described herein. In one example, the carrier includes all or a portion of the local anesthetic agent and includes a commercially available medicated local anesthetic composition comprising a cream, ointment, suspension, lotion, gel, or solution. For example, the carrier may comprise a commercially available medicated Lidocaine Ointment, Lidocaine Cream, Lidocaine and Prilocaine Cream, or Lidocaine Solution. In one instance, a method of making the topical composition comprises combining the antimicrobial agent with a Lidocaine Solution including lidocaine in an aqueous solution. The lidocaine solution may be a commercially available lidocaine topical solution, such as lidocaine hydrochloride solution for topical administration. The carrier may comprise the lidocaine solution. The lidocaine hydrochloride solution may contain, for example, 4% lidocaine (w/v) wherein each mL includes 40 mg lidocaine HCl. For example, in one embodiment, the lidocaine topical solution may be Lidocaine Hydrochloride Topical Solution USP, 4% manufactured by IGI Labs, Inc., Buena, N.J., in 50 mL screw cap glass bottles. The lidocaine hydrochloride topical solution may contain various inactive ingredients such as methylparaben, purified water, and sodium hydroxide to adjust pH to 6.0-7.0.

In one embodiment, the method of formulating the topical composition comprises combining an antimicrobial agent, carrier, and a steroid agent. In one example, the steroid agent comprises a corticosteroid selected from amcinonide, betamethasone dipropionate, betamethasone valerate, clobetasol propionate, desoximetasone, diflorasone diacetate, flurandrenolide, fluticasone propionate, fluocinonide, halcinonide, halobetasol propionate, mometasone furoate, triamcinolone acetonide, or combination thereof. In another example, the steroid agent comprises a corticosteroid selected from betamethasone dipropionate, betamethasone valerate, clobetasol propionate, fluticasone propionate, fluocinonide, halcinonide, halobetasol propionate, or combination thereof In various embodiments, the topical composition comprises between approximately 0.001% and approximately 1% by weight steroid agent.

In one embodiment, the method of formulating the topical composition comprises combining the antimicrobial agent, a steroid agent identified herein, and a carrier in an amount sufficient to formulate the topical composition comprising the antimicrobial agent in an amount between approximately 0.01% and approximately 10% by weight, such as between approximately 0.5% and approximately 5% or any other percent, percent range, or percent therebetween by weight described herein and the steroid agent in an amount between approximately 0.01% and approximately 2% by weight, such as between approximately 0.05% and approximately 1% or any other percent, percent range, or percent therebetween by weight described herein. The antimicrobial agent may be or include any antibacterial or antifungal active described herein. For example, in one embodiment, the antimicrobial agent comprises an antifungal agent selected from fluconazole, voriconazole, or combination thereof. In one embodiment, the method includes crushing voriconazole 50 mg, 100 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the antimicrobial agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg tablets and the carrier. Combining the steroid agent may comprise adding a bulk powder, crushed tablet, or injection powder. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one embodiment, the carrier comprises a commercially available medicated composition comprising a portion of the antimicrobial agent or an additional active agent as described herein. In one example, the carrier includes all or a portion of the steroid agent and includes a commercially available medicated steroid composition comprising a cream, ointment, suspension, lotion, gel, or solution. For example, the carrier may comprise a commercially available Clobetasol Propionate Cream, Foam, Gel, or Ointment, Diflorasone Diacetate Cream or Ointment, Amcinonide Cream, Lotion, or Ointment, Betamethasone Dipropionate Cream, Lotion, Gel, or Ointment, Desoximetasone Cream or Ointment, Fluocinonide Cream, Fluocinonide Cream, Ointment, or Gel, Halcinonide Cream or Ointment, Fluocinolone Acetonide Cream, Ointment, Oil, or Solution, Halcinonide Cream or Ointment, Betamethasone Valerate Cream, Lotion, or Ointment, Diflorasone Diacetate Cream or Ointment, Triamcinolone Acetonide Cream or Ointment, Halobetasol Propionate Cream, Lotion, or Ointment, Desoximetasone Cream, Gel, or Ointment, Mometasone Furoate Cream or Ointment, Fluticasone Propionate Cream, Flurandrenolide Cream, Lotion, or Ointment, or combination thereof. In another example, the corticosteroid topical composition is selected from Clobetasol Propionate Cream or Ointment, Diflorasone Diacetate Cream or Ointment, Amcinonide Cream or Ointment, Betamethasone Dipropionate Cream or Ointment, Desoximetasone Cream or Ointment, Fluocinonide Cream or Ointment, Fluocinolone Acetonide Cream, Ointment, Oil, or Solution, Halcinonide Cream or Ointment, Triamcinolone Acetonide Cream or Ointment, Halobetasol Propionate Cream or Ointment, Mometasone Furoate Cream or Ointment, Flurandrenolide Cream or Ointment, or combination thereof. In still another example, the carrier comprises Clobetasol Propionate Cream or Ointment, Fluocinonide Cream or Ointment, Fluocinolone Acetonide Cream, Ointment, Oil, or Solution, Halcinonide Cream or Ointment, Halobetasol Propionate Cream, or Desoximetasone Cream or Ointment, Triamcinolone Acetonide Cream or Ointment, Betamethasone Dipropionate Cream or Ointment, or combination thereof. In another example, the carrier comprises Clobetasol Propionate Cream, Foam, Gel, or Ointment, 0.05%, Diflorasone Diacetate Cream or Ointment; 0.05%, Amcinonide Cream, Lotion, or Ointment, 0.1%, Betamethasone Dipropionate Cream Lotion, Gel, or Ointment 0.05%, Desoximetasone Cream or Ointment 0.25%, Fluocinonide Cream 0.1%, Fluocinonide Cream, Ointment, or Gel, 0.05%, Fluocinolone Acetonide Cream 0.01%, Fluocinolone Acetonide Cream 0.025%, Fluocinolone Acetonide Oil 0.01%, Fluocinolone Acetonide Ointment 0.01%, Fluocinolone Acetonide Ointment 0.025%, Fluocinolone Acetonide Solution 0.01%, Halcinonide Cream or Ointment, 0.1%, Betamethasone Valerate Cream, Lotion, or Ointment 0.1%, Diflorasone Diacetate Cream or Ointment, 0.05%, Triamcinolone Acetonide Cream or Ointment, 0.1%, Triamcinolone Acetonide Ointment, 0.05%, Halobetasol Propionate Cream, Lotion, or Ointment, 0.05%, Desoximetasone Cream, Gel, or Ointment 0.05%, Mometasone Furoate Cream or Ointment, 0.1%, Fluticasone Propionate Cream, 0.05%, Flurandrenolide Cream, Lotion, or Ointment, 0.05%, or combination thereof. In still a further example of the above, the corticosteroid topical composition is selected from Clobetasol Propionate Cream or Ointment, 0.05%, Diflorasone Diacetate Cream or Ointment, 0.05%, Amcinonide Cream or Ointment, 0.1%, Betamethasone Dipropionate Cream or Ointment 0.05%, Desoximetasone Cream or Ointment 0.25%, Fluocinonide Cream 0.1%, Fluocinonide Cream or Ointment, 0.05%, Fluocinolone Acetonide Cream 0.01%, Fluocinolone Acetonide Cream 0.025%, Fluocinolone Acetonide Oil 0.01%, Fluocinolone Acetonide Ointment 0.01%, Fluocinolone Acetonide Ointment 0.025%, Fluocinolone Acetonide Solution 0.01%, Halcinonide Cream or Ointment, 0.1%, Diflorasone Diacetate Cream or Ointment, 0.05%, Triamcinolone Acetonide Cream, 0.1%, Halobetasol Propionate Cream or Ointment, 0.05%, Desoximetasone Cream or Ointment 0.05%, Mometasone Furoate Cream or Ointment, 0.1%, or Flurandrenolide Cream or Ointment, 0.05%, or combination thereof. In still a further embodiment, the corticosteroid topical composition is selected from Betamethasone Dipropionate Cream or Ointment 0.05%, Clobetasol Propionate Cream or Ointment, 0.05%, Desoximetasone Cream or Ointment 0.25%, Fluocinonide Cream 0.1%, Fluocinonide Cream or Ointment, 0.05%, Fluocinolone Acetonide Cream 0.01%, Fluocinolone Acetonide Cream 0.025%, Fluocinolone Acetonide Oil 0.011%, Fluocinolone Acetonide Ointment 0.01%, Fluocinolone Acetonide Ointment 0.025%, Fluocinolone Acetonide Solution 0.01%, Triamcinolone Acetonide Cream, 0.1%, Halobetasol Propionate Cream, 0.05%, or Desoximetasone Cream or Ointment 0.05%, or combination thereof.

In one embodiment, the method of formulating the topical composition comprises combining an antimicrobial agent, carrier, and an additional active agent selected from one or more muscle relaxants, anticonvulsants, nerve depressants, NMDA receptor antagonists, opiates, opioid agonists, or combinations thereof. A muscle relaxant agent may comprise between approximately 0.001% and approximately 5% by weight of the topical composition and be selected from baclofen, carisoprodol, chlorzoxazone, cyclobenzaprine, dantrolene, diazepam, metaxalone, methocarbamol, orphenadrine, quinine sulfate, tizanidine, or combination thereof; an anticonvulsant or nerve depressant agent may comprise between approximately 0.01% and approximately 20% by weight of the topical composition and be selected from gabapentin, topiramate, lamotrigine, or combinations thereof; a NMDA receptor antagonist agent may include ketamine; an opiate or opioid agonist agent may include tramadol, oxycodone, morphine, methadone, hydromorphone, fentanyl, hydrocodone, codeine, propoxyphene, butalbital, pentazocine, or combination thereof. The antimicrobial agent may be combined in an amount between approximately 0.01% and approximately 10% by weight of the topical composition, such as between approximately 0.5% and approximately 5% or any other percent, percent range, or percent therebetween by weight described herein. The antimicrobial agent may be or include any antibacterial or antifungal active described herein. For example, in one embodiment, the antimicrobial agent comprises an antifungal agent selected from fluconazole, voriconazole, or combination thereof. For example, in one embodiment, the antimicrobial agent comprises an antifungal agent selected from fluconazole, voriconazole, or combination thereof. In one embodiment, the method includes crushing voriconazole 50 mg, 100 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the antimicrobial agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg tablets and the carrier. Combining the additional active agent may comprise adding a bulk powder, crushed tablet, or injection powder. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one embodiment, the carrier comprises a commercially available medicated composition comprising a portion of the antimicrobial agent or an additional active agent as described herein. In one example, the carrier includes all or a portion of the additional active agent and includes a commercially available medicated composition comprising the additional active agent in a cream, ointment, suspension, lotion, gel, or solution.

In one embodiment, the method of formulating the topical composition comprises combining an antimicrobial agent; carrier, and a keratolytic agent. The keratolytic agent selected form urea, salicylic acid, papain, or combinations thereof. For example, the topical composition may comprise the antimicrobial agent and urea. In various embodiments, the topical composition may comprise between approximately 1% and approximately 30% by weight urea.

In one embodiment, the method of formulating the topical composition comprises combining the antimicrobial agent, a keratolytic agent identified herein, and a carrier in an amount sufficient to formulate the topical composition comprising the antimicrobial agent in an amount between approximately 0.01% and approximately 10% by weight, such as between approximately 0.5% and approximately 5% or any other percent, percent range, or percent therebetween by weight described herein and the keratolytic agent in an amount between approximately 5% and approximately 30% by weight, such as between approximately 10% and approximately 20% or any other percent, percent range, or percent therebetween by weight described herein. The antimicrobial agent may be or include any antibacterial or antifungal active described herein. For example, in one embodiment, the antimicrobial agent comprises an antifungal agent selected from fluconazole, voriconazole, or combination thereof. In one embodiment, the method includes crushing voriconazole 50 mg, 100 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the antimicrobial agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg tablets and the carrier. Combining the keratolytic may comprise adding a bulk powder, crushed tablet, e.g., crushed urea tablet, or injection powder. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one embodiment, the carrier comprises a commercially available medicated composition comprising a portion of the antimicrobial agent or an additional active agent as described herein. In one example, the carrier includes all or a portion of the keratolytic agent and includes a commercially available medicated keratolytic comprising a urea ointment or cream. For example, the carrier may comprise REA LO 40®, which is a 40.0% urea cream. Each gram of REA LO 40® contains 400 mg urea as the active ingredient and the following inactive ingredients: purified water, emulsifying wax, glycerin, isopropyl myristate, sorbitol, neopentyl glycol dicaprylate/dicaprate, tridecyl stearate, tridecyl trimellitate and dimethyl isosorbide. The urea cream may comprise various percentages of urea by weight (prior to compounding or prior to combination with another carrier or carrier component), such as 10%, 15%, 20%, 25%, 30%, 35%, 40%, or any other commercially available percentage by weight. In various embodiments, the urea cream may be Urix 40 Urea Cream marketed by Topix Pharmaceuticals, Inc. Urix 40 Urea Cream includes 40% urea or 400 mg urea per gram and further includes Carbomer, Cyclomethicone, Dimethicone Silyate, Dimethiconol, Glycerin, Hydrogenated Lecithin, Imidazolidinyl Urea, Petrolatum, Phenyl. Trimethicone, Polyphosphorylcholine Glycol Acrylate, Triethanolmine, Water, and Xanthan Gum. In additional embodiments, the urea cream may be Rea Lo 40 topical or Rea Lo 30 topical marketed by Crown Laboratories. Rea Lo 40 topical comprises 400 mg urea per gram and Rea Lo 30 topical comprises 300 mg urea per gram. Rea Lo 40 topical and Rea Lo 30 topical further include purified water, emulsifying wax, glycerin, isopropyl myristate, sorbitol, neopentyl, glycol dicaprylate/dicaprate, tridecyl stearate, tridecyl trimellitate and dimethyl isosorbide. In additional embodiments, the urea cream may be Urea 10% Cream by Stratus Pharmaceuticals, Inc. Urea 10% Cream includes 10% urea or 100 mg urea per gram, and further includes Carbomer, Fragrance, Isopropyl Myristate, Isopropyl Pahnitate, Propylene Glycol, Purified Water, Sodium Laureth Sulfate, Stearic Acid, Trolamine and Xanthan Gum. It is to be understood that the above urea creams (or any other urea cream) may be diluted or cut prior to or, in some embodiments, after compounding or otherwise combining the urea cream with additional creams and/or actives. Thus, the topical composition may comprise less urea by weight than was present in the urea cream prior to compounding or combination with another cream and/or active.

The method of formulating a topical composition may comprise combining an antimicrobial agent comprising crushed oral tablets and a carrier. In one example, the antimicrobial agent comprises an antifungal agent comprising voriconazole and the method of formulating the topical composition comprises addition of a crushed voriconazole tablet to a carrier. The voriconazole tablets may comprise commercially available voriconazole 50 mg, 100 mg, 200 mg oral tablets. The oral tablets may be crushed and combined with the carrier to formulate a topical composition comprising between 0.01% and 20% by weight, such as approximately 1%, approximately 2%, approximately 3%, approximately 4%, approximately 5%, less than approximately 5%, between approximately 2% and approximately 7%, or greater than approximately 10% voriconazole by weight. To formulate a topical composition comprising a desired percent by weight voriconazole, the total desired weight of the topical composition is subtracted from the weight of crushed oral voriconazole tablet powder needed to obtain the desired percent by weight voriconazole. The weight of voriconazole tablet powder needed is determined by multiplying the weight of active needed to obtain the desired percent by weight voriconazole in the topical composition. For example, a topical composition comprising 1% voriconazole may be formulated combining powder obtained from 200 mg oral voriconazole tablets. The weight of voriconazole tablet powder needed is determined by multiplying the weight of voriconazole needed to obtain the desired percent by weight voriconazole in the topical composition. Here, a 1% voriconazole composition includes 10 mg voriconazole per gram. If a 200 mg voriconazole tablet weights approximately 450 mg, 22.5 mg of crushed voriconazole tablet powder comprises 10 mg voriconazole. Therefore, 22.5 mg of crushed voriconazole tablet powder is combined for each gram of topical composition. Consequently, 977.5 mg of carrier, carrier components thereof, and additional active agents, if any, may be combined with 22.5 mg of crushed voriconazole tablet powder to formulate each gram of topical composition to formulate a 1% by weight topical composition. Other percent compositions may be formulated as described herein. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional active agents may include one or more antifungal actives, antibacterial actives, or both. Such additional antimicrobial agent may be present in a combined amount between 0.01% and 20% by weight, such as between approximately 0.01% and approximately 5%. Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0,01% and 25% by weight, such as between approximately 1% and approximately 10%.

In one example, the antimicrobial agent comprises an antifungal agent comprising fluconazole and the method of formulating the topical composition comprises addition of a crushed fluconazole tablet to a carrier. The fluconazole tablets may comprise commercially available fluconazole 100 mg and/or 200 mg oral tablets. The oral tablets may be crushed and combined with the carrier to formulate a topical composition comprising between 0.01% and 20% by weight, such as approximately 1%, approximately 2%, approximately 3%, approximately 4%, approximately 5%, less than approximately 5%, between approximately 2% and approximately 7%, or greater than approximately 10% fluconazole by weight. To formulate a topical composition comprising a desired percent by weight fluconazole, the total desired weight of the topical composition is subtracted from the weight of crushed oral fluconazole tablet powder needed to obtain the desired percent by weight fluconazole in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional active agents may include one or more antifungal actives, antibacterial actives, or both. Such additional antimicrobial agent may be present in a combined amount between 0.01% and 20% by weight, such as between approximately 0.01% and approximately 5%. Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0.01% and 25% by weight, such as between approximately 1% and approximately 10%.

In one example, the antimicrobial agent comprises an antibacterial agent comprising linezolid and the method of formulating the topical composition comprises addition of a crushed linezolid tablet to a carrier. The linezolid tablets may comprise commercially available linezolid 600 mg oral tablets. The oral tablets may be crushed and combined with the carrier to formulate a topical composition comprising between 0.01% and 20% by weight, such as approximately 1%, approximately 2%, approximately 3%, approximately 4%, approximately 5%, less than approximately 5%, between approximately 2% and approximately 7%, or greater than approximately 10% linezolid by weight. To formulate a topical composition comprising a desired percent by weight linezolid, the total desired weight of the topical composition is subtracted from the weight of crushed oral levofloxacin tablet powder needed to obtain the desired percent by weight linezolid in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional active agents may include one or more antifungal actives, antibacterial actives, or both. Such additional antimicrobial agent may be present in a combined amount between 0.01% and 20% by weight, such as between approximately 0.01% and approximately 5%. Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0.01% and 25% by weight, such as between approximately 1% and approximately 10%.

In one example, the antimicrobial agent comprises an antibacterial agent comprising levofloxacin and the method of formulating the topical composition comprises addition of a crushed levofloxacin tablet to a carrier. The levofloxacin tablets may comprise commercially available levofloxacin 250 mg, 500 mg, 750 mg oral tablets. The oral tablets may be crushed and combined with the carrier to formulate a topical composition comprising between 0.01% and 20% by weight, such as approximately 1%, approximately 2%, approximately 3%, approximately 4%, approximately 5%, less than approximately 5%, between approximately 2% and approximately 7%, or greater than approximately 10% levofloxacin by weight. To formulate a topical composition comprising a desired percent by weight levofloxacin, the total desired weight of the topical composition is subtracted from the weight of crushed oral levofloxacin tablet powder needed to obtain the desired percent by weight levofloxacin in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional active agents may include one or more antifungal actives, antibacterial actives, or both. Such additional antimicrobial agent may be present in a combined amount between 0.01% and 20% by weight, such as between approximately 0.01% and approximately 5%. Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0.01% and 25% by weight, such as between approximately 1% and approximately 10%.

In one example, the antimicrobial agent comprises an antibacterial agent comprising ciprofloxacin and the method of formulating the topical composition comprises addition of a crushed ciprofloxacin tablet to a carrier. The ciprofloxacin tablets may comprise commercially available ciprofloxacin 250 mg, 500 mg, 750 mg oral tablets. The oral tablets may be crushed and combined with the carrier to formulate a topical composition comprising between 0.01% and 20% by weight, such as approximately 1%, approximately 2%, approximately 3%, approximately 4%, approximately 5%, less than approximately 5%, between approximately 2% and approximately 7%, or greater than approximately 10% ciprofloxacin by weight. To formulate a topical composition comprising a desired percent by weight ciprofloxacin, the total desired weight of the topical composition is subtracted from the weight of crushed oral ciprofloxacin tablet powder needed to obtain the desired percent by weight ciprofloxacin in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional active agents may include one or more antifungal actives, antibacterial actives, or both. Such additional antimicrobial agent may be present in a combined amount between 0.01% and 20% by weight, such as between approximately 0.01% and approximately 5%. Additionally or alternatively, additional actives may include other active agents such as one or more active agents selected from an antiviral agent, an anti-inflammatory agent, a steroid, an anti-allergic agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0.01% and 25% by weight, such as between approximately 1% and approximately 10%.

### Method of Treating an Infection

The present disclosure also describes methods of treating an infection by providing or administering a topical composition described herein. The method may include contacting the topical composition to skin, nails, or body orifice that is infected or believed to be infected. The infection may be of an exterior surface of the body, an orifice, or internal. Administration may include bath-irrigation, topical irrigation via a syringe, administration in a topical powder, or a topical gel, cream, ointment, or lotion. Administration may be to an external surface of the body or to anal or vaginal surfaces. In various embodiments, the topical composition may be administered via contact to an infected area such as to skin of a head, face, ears, nose, neck, shoulder, torso, chest, stomach, waistline, extremity, arm, hand, finger, nail, groin, buttock, leg, foot, or toe, for example. In an embodiment of a method to treat an internal infection, the topical composition may be administered topically as described herein wherein one or more active agents are transdermally delivered locally or for systemic circulation. Additional active agents may be utilized in the topical composition to reduce pain, irritation, and inflammation such as NSAIDs, steroids, local anesthetics, anticonvulsants, antidepressants, for example. In various embodiments, the topical composition may be administered 1 to 2 times daily or as otherwise needed.

In one embodiment, a topical composition may be used to treat an infection or suspected infection accompanying a hyperkeratotic skin conditions that are marked by a thickening of the outer layers of skin. Methods of using the topical composition may include treating an individual in need by topically applying the composition to affected skin. Conditions treated may include conditions such as those marked by thickening of the skin, referred to as hyperkeratosis. The compounded topical composition described herein may thus be applied to such affected areas of the skin to treat the affected area. The composition may alleviate symptoms such as redness, swelling, or itching. The composition may accelerate the healing process with respect to the affected skin. In various embodiments, the topical composition may be administered to treat hyperkeratotic conditions. The hyperkeratotic skin condition treated may include chronic eczema, corns, calluses, warts, seborrheic keratosis, lichen planus, actinic keratosis, as examples. The hyperkeratotic skin conditions may be caused by irritation, such as physical pressure or rubbing, chemical, infection, sunlight or radiation, or inherited conditions, for example. In an embodiment, the topical composition may be administered to such affected skin in a preventative treatment regime to combat proliferation of microbial infections with respect to the thickened skin layers. In some such embodiments, the topical composition may include a keratolytic agent as described herein.

Topical compositions comprising cream, lotion, paste, ointment, and similar formats may be applied by contact to skin, or mucosal tissue with respect to anal or vaginal administration. In some embodiments, the topical composition may be formulated in a shampoo carrier for administration in a shampoo. In some formats, the composition may be administered to an infected or target area via spray, drops, wash, swab, sponge, absorbent dressing, coating (e.g., a nail lacquer), soaking, submerging, footbath, instillation or irrigation. Embodiments comprising a nail lacquer formulation may be applied directly to nails, to treat a bacterial or fungal nail infection.

Various embodiments comprising a solution format may be administered in a footbath, which may include a hand bath or soak, to treat or prevent an infection. The method may include adding the topical composition to a footbath. In some embodiments, the method may include addition of a carrier or carrier component comprising an aqueous diluent. The aqueous diluent may be in addition to the carrier as described herein or may be the carrier. For example, a. topical composition comprising a solution, cream, ointment, powder, gel paste, or lotion format may be added to a footbath. Additional carrier comprising an aqueous diluent may also be added. In some embodiments, the topical composition prior to addition of the diluent comprises a concentrated topical composition, and following addition of the carrier component comprising the diluent, the topical composition comprises the percent compositions described herein. The footbath solution may be agitated and/or heated in some embodiments. A foot or a hand may contact the footbath solution in the footbath for administration of the topical composition.

A “footbath” refers to a container that can hold some volume (e.g., about 1.0 liters to about 10 liters) of a footbath solution, which may typically be an aqueous solution or suspension, and is designed to physically accommodate at least a portion of one or both feet of a subject. A footbath administration includes administration of the topical composition utilizing a footbath. A footbath may be used as a hand bath; however, smaller bathing containers may typically be utilized as hand baths. In various embodiments, footbath solutions may be utilized as hand bath solutions. A footbath can comprise several features or agents that effect various functions. For example, a footbath can comprise one or more lights or light-emitting devices, a mechanical agitation agent (e.g., one or more jets or bubble makers) to physically agitate the enclosed water, a bubble agent to create bubbles within the enclosed water, a heating agent to heat the enclosed water, a vibration agent to vibrate the enclosed water (e.g., a high frequency vibration massage), an infrared device to provide infrared light to a foot or feet of the subject, a massage agent (e.g., a roller) that provides massaging contact to at least a portion of one or both feet, a pedicure agent that can clean or contact a foot or feet with a pumice, or a combination thereof. In an aspect, a footbath can have a water fall element. In an aspect, an agitation agent or an agitator can be coupled to both a motor and the footbath. In an aspect, a footbath can comprise one or more splash guards and other spill-resistant features to ensure that the water remains enclosed within a container. A footbath may also accommodate a subject's calves, meaning that the container is “deep” so as to allow the enclosed water to contact both the feet and at least a portion of the calves of the subject. Several manufacturers market footbaths including PIBB, Dr. Scholl's, Kendal, Conair (e.g., Model FB5X, FB3, FB27R, FB30, FB52, etc.), and Brookstone.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

Various elements described herein have been described as alternatives or alternative combinations, e.g., in a lists of selectable actives, ingredients, or compositions. It is to be appreciated that embodiments may include one, more, or all of any such elements. Thus, this description includes embodiments of all such elements independently and embodiments including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “approximately” are intended to include +/−10% of the number modified.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. A method of formulating a topical composition, the method comprising:
combining:
a powder comprising an antimicrobial pharmaceutical drug; and
a topical carrier,
wherein the powder comprises at least a portion of a crushed oral tablet comprising the antimicrobial pharmaceutical drug, and wherein the antimicrobial pharmaceutical drug is selected from voriconazole, fluconazole, linezolid, levofloxacin, ciprofloxacin, or combination thereof.

2. The method of claim 1, further comprising crushing the oral tablet comprising the antimicrobial pharmaceutical drug.

3. The method of claim 2, wherein the oral tablet comprises a voriconazole 50 mg, 100 mg, or 200 mg oral tablet.

4. The method of claim 2, wherein the oral tablet comprises a fluconazole 100 mg or 200 mg oral tablet.

5. The method of claim 2, wherein the oral tablet comprises a linezolid 600 mg oral tablet.

6. The method of claim 2, wherein the oral tablet comprises a ciprofloxacin 250 mg, 500 mg, or 750 mg oral tablet.

7. The method of claim 2, wherein the oral tablet comprises a levofloxacin 250 mg, 500 mg, or 750 mg oral tablet.

8. The method of claim 1, wherein the topical carrier comprises a solution, cream, ointment, gel, or foam.

9. The method of claim 8, wherein the topical carrier comprises a commercially available medicated composition comprising at least one additional active pharmaceutical drug.

10. The method of claim 2, further comprising removing a film coating on the oral tablet prior to crushing the oral tablet comprising agitating the oral tablet for approximately 5 to approximately 20 seconds while the oral tablet is contacted with a solvent, and, thereafter, removing the solvent from contact with the oral tablet.

11. A method of treating a bacteria or fungal infection, the method comprising:
topically administering a composition comprising at least a portion of an oral tablet combined with a carrier to an affected external skin or nail region or an anal or vaginal orifice,
wherein the oral tablet comprises an antimicrobial pharmaceutical drug selected from voriconazole, fluconazole, linezolid, levofloxacin, ciprofloxacin, or combination thereof.

12. The method of claim 11, wherein the composition comprises a solution, and wherein topically administering the composition comprises administering to skin or a nail of a foot or hand in a footbath.

13. The method of claim 11, wherein the composition comprises an irrigation solution, and wherein topically administering the composition comprises irrigating the affected external skin or nail region or anal or vaginal orifice.

14. The method of claim 11, wherein the composition comprises a nail lacquer solution, wherein and topically administering the composition comprises applying the nail lacquer solution to a nail of a foot or hand.

15. The method of claim 11, further comprising formulating the composition, wherein formulating the composition comprises combining:
a powder comprising at least a portion of the oral tablet having been crushed to generate the powder; and
the carrier.

16. The method of claim 15, wherein the oral tablet comprises a voriconazole 50 mg oral tablet, voriconazole 100 mg oral tablet, voriconazole 200 mg oral tablet, fluconazole 100 mg oral tablet, fluconazole 200 mg oral tablet, or combination thereof.

17. The method of claim 15, wherein the oral tablet comprises a linezolid 600 mg oral tablet, ciprofloxacin 250 mg oral tablet, ciprofloxacin 500 mg oral tablet, ciprofloxacin 750 mg oral tablet, levofloxacin 250 mg oral tablet, levofloxacin 500 mg oral tablet, levofloxacin 750 mg oral tablet, or combination thereof.

18. The method of claim 15, wherein the carrier comprises a cream, ointment, gel, foam, or powder.

19. The method of claim 18, wherein the carrier comprises a commercially available medicated composition comprising at least one additional active pharmaceutical drug.

20. The method of claim 15, further comprising:
crushing the oral tablet; and
removing a film coating on the oral tablet prior to crushing the oral tablet, wherein removing the film coating comprises agitating the oral tablet for approximately 5 to approximately 20 seconds while the oral tablet is contacted with a solvent, and, thereafter, removing the solvent from contact with the oral tablet.
